(19) United States
(12) Patent Application Publication
(10) Pub. No.: US 2013/0191145 A1
(43) Pub. Date: Jul. 25, 2013

(54) SYSTEM AND APPARATUS FOR GENERATING WORK SCHEDULES

(71) Applicant: ClearCare, Inc., San Francisco, CA (US)

(72) Inventors: Geoffrey Howard Nudd, San Francisco, CA (US); Peter Robert Sheats, Largo, FL (US); Anthony J. Ina, JR., Brooklyn, NY (US)

(73) Assignee: ClearCare, Inc., San Francisco, CA (US)

(21) Appl. No.: 13/767,421

(22) Filed: Feb. 14, 2013

Related U.S. Application Data
(63) Continuation-in-part of application No. 13/625,718, filed on Sep. 24, 2012.
Application No. 13/625,718 is a continuation-in-part of application No. 13/571,305, filed on Aug. 9, 2012.
Application No. 13/571,305 is a continuation-in-part of application No. 13/494,901, filed on Jun. 12, 2012.
Application No. 13/494,901 is a continuation-in-part of application No. 13/444,737, filed on Apr. 11, 2012.
Application No. 13/444,737 is a continuation-in-part of application No. 13/420,506, filed on Mar. 14, 2012.
Application No. 13/420,506 is a continuation-in-part of application No. 13/293,039, filed on Nov. 9, 2011.
Application No. 13/293,039 is a continuation-in-part of application No. 13/277,146, filed on Oct. 19, 2011.
Application No. 13/277,146 is a continuation-in-part of application No. 13/180,447, filed on Jul. 11, 2011.
(60) Provisional application No. 61/474,145, filed on Apr. 11, 2011.
(60) Provisional application No. 61/452,443, filed on Mar. 14, 2011.
(60) Provisional application No. 61/394,676, filed on Oct. 19, 2010.

(51) Int. Cl. G06Q 10/06; G06Q 50/22

(52) U.S. Cl. CPC G06Q 10/063118; G06Q 50/22
USPC 705/2

(57) ABSTRACT

Disclosed are new approaches for scheduling workers at remote locations. Work assignments may be organized into shifts associated with one or more workers and one or more recipients. Tasks are associated with the shift and have an updatable status. Shifts may be replicated by cutting and pasting or by specifying a recurrence definition. The tasks associated with a shift may then be replicated. The status of tasks may be updated using a voice telephony system. The status of tasks may also be reported from a computer located on the recipient premise. Special classification activities may also be automatically specified according to rules and associated with shifts and replicated shifts. Methods for automatically associating providers with shifts are also disclosed. A family portal enabling access and provisional changes to care data for a recipient is also disclosed.

### RELATED APPLICATIONS

This application is a continuation-in-part of U.S. application Ser. No. 13/625,718 filed Sep. 24, 2012, which is hereby incorporated herein in its entirety.

This application is a continuation-in-part of U.S. application Ser. No. 13/571,305 filed Aug. 9, 2012, which is hereby incorporated herein in its entirety.

This application is a continuation-in-part of U.S. application Ser. No. 13/494,901 filed Jun. 12, 2012, which is hereby incorporated herein in its entirety.

This application is a continuation-in-part of U.S. application Ser. No. 13/444,737 filed Apr. 11, 2012, which claims the benefit of U.S. Provisional Application Ser. No. 61/474,145, both of which Applications are hereby incorporated herein in their entirety.

This application is a continuation-in-part of U.S. application Ser. No. 13/420,506 filed Mar. 14, 2012, which claims the benefit of U.S. Provisional Application Ser. No. 61/452,443, both of which Applications are hereby incorporated herein in their entirety.

This application is a continuation-in-part of U.S. application Ser. No. 13/293,039 filed Nov. 9, 2011, which is hereby incorporated herein in its entirety.

This application is a continuation-in-part of U.S. application Ser. No. 13/277,146 filed Oct. 19, 2011, which claims the benefit of U.S. Provisional Application No. 61/394,676, both of which Applications are hereby incorporated herein by reference in their entirety.

This application is a continuation-in-part of U.S. application Ser. No. 13/180,447 filed Jul. 11, 2011, which is hereby incorporated herein in its entirety.

### BACKGROUND

Computer-enabled calendar systems date to the early days of software. In the 1990s and thereafter, a growing number of online calendar systems have been introduced which enable a user to, among other functions, create new events and tasks, schedule with other users, and send and receive reminders. Many of these calendars are now available online, such as that provided by Google Calendar, which access across geographies via any Internet-enabled terminal. A problem with existing online calendar systems is in their management of “tasks,” which may be defined as an assignment of work to-be-completed with an assigned date on which the work is to be completed and/or started and/or in-progress, and at least one complete or incomplete state.

As defined herein, tasks are a superset which contains “events” which are typically meetings or scheduled occurrences in which the work to-be-completed primarily or exclusively involves attendance or participation in the event itself (i.e. a meeting). An important differentiator between events and tasks which are not events, which are often referred to as “to do's” and which we shall call “nonevent tasks”, is that non-event tasks lend themselves to tracking via checklists, a well known and remarkably effective and simple way to track outstanding and completed tasks, wherein it is generally not effective or useful to track events via checklists (i.e. a checklist of outstanding and/or completed meetings).

A subtle but important oversight is that the existing online calendar systems such as Google Calendar, Yahoo Calendar and others have built rich functional capabilities for the management of events such as the ability to create recurring series of events (i.e. a meeting that occurs every Monday at 10:00 AM) or the ability to send invitations to a variety of attendees, but have not introduced similar capabilities for the management of non-event tasks. Conversely, existing calendar systems have introduced functionality such as checklists for non-event tasks that have not been created for events. This introduces a significant shortcoming, particularly in the creation of work management systems that provide the ease of use and flexibility of a calendar interface with the work tracking capabilities of checklists.

In particular, the inventor finds that it is an important shortcoming of the existing art that no existing online calendar systems enable the ability to create recurring non-event tasks in a computer-enabled system with a checklist interface that enables a user to mark the status of a task (including but not limited to marking the status of a task as complete).

These problems become acute in the use of electronic calendaring and scheduling systems for the management of remote workers. Additionally, there is a shortcoming in the known art in which it is time consuming to specify a group of tasks, opposed to individual tasks, which are recurring on a schedule. It would be desirable to have means by which a set and/or group of tasks could be defined for a time period in which the entire set could be made recurring according to some criteria. It would furthermore be desirable if said tasks could be managed and monitored without regard to geographical limitations via means including but not limited to checklists.

Moreover, today, computer-enabled online calendar systems are only accessible via a computer terminal with a visual interface such as a computer monitor and require some form of Internet connection. As there are today no means of creating recurring non-event tasks in a calendar system and managing their completion via a checklist interface, it follows that there are no means of interfacing with said new inventive systems via any means. It would be advantageous if the functionality could be accessible by a remote computer terminal connected to the Internet. Moreover, for situations in which a remote computer terminal connected to the Internet is difficult or cost prohibitive, it would be advantageous if there were other means to interface with said inventive online calendar functionality. The invention described herein will address these problems.

While there exists simple clock-in and clock-out functionality via telephony relative to expected work times and/or times of worker's shift, such as that provided by Santrax (www.santrax.com), there is presently no way to access such calendar systems with task-level specificity via telephony. Solutions such as Santrax have existed for many years without solving the problem of task-level specificity, nor have they solved the aforementioned problems with the treatment of non-event tasks. These are critical oversights that significantly reduce the usefulness of the known art.

By way of example and without limitation, in the in-home health care industry, solutions like Santrax are used to track clock-in and clock-out times relative to shifts using telephony to update the clock-in or clock-out status of a remote caregiver. While this system enables specification of work shifts and remote updates of clock-in and clock-out status, the detailed tasks that comprise a care plan cannot be scheduled relative to a shift and updated via the remote telephony system. Instead, the only way tasks can be tracked is wherein the remote worker enters codes via the telephone wherein the codes correspond to tasks. This has the disadvantage that tasks that are not completed cannot be tracked, tasks cannot be managed relative to a shift schedule, and comments cannot be provided by the remote worker to provide critical information relative to the status of the task. There are complex challenges associated with enabling such an improved system, such as text-to-voice automated translation of tasks in a care plan, which heretofore have not been solved. Additionally, again considering without limitation the present example of in-home care agency management software, today there does not exist a flexible, easy-to-use calendar system that enables the specification of non-event tasks with features like recurrence of an event at specific times during specific days of the week, weeks in the month, etc. and the ability to update status in an easy-to-use electronic checklist.

To have such a system would provide flexibility and ease-of-use that today does not exist for the service of the in-home care agency industry. By way of example, software systems such as that provided by HomeTrak (www.hometrak.us) enable the specification of recurring events or “shifts” on an online calendar system for the in-home care agency industry, but do not allow the specification of non-event tasks related to the shift. As such, while HomeTrak can verify that a remote caregiver has arrived at the home of a patient, they cannot perform more detailed tracking of non-event task completion.

These shortcomings with the existing art lead to many problems including very limited transparency and control over the care plan to stakeholders such as in-home care managers, healthcare providers, and the family members of a patient or client. Moreover, in the example of the in-home care industry, these shortcomings today are addressed via mechanisms like paper care journals which reside in the home of the patient and which are periodically updated by caregivers. The paper care journals are often overlooked by caregivers and the in-home care managers, and the families of the patients have no visibility to the care provided and the tasks performed. Moreover, even if the paper care journals are completed, they must be collected from the homes of patients by the care agency, and because the records can be lengthy, problems of storage arise.

This industry example illustrates the very significant and important problems with the existing art, and the quality of care can be significantly improved by solving these problems. The present invention solves these problems thus enabling work management systems with unprecedented ease of use, flexibility, and cost-effective accessibility in a plurality of locations that was never before possible.

### DESCRIPTION OF THE DRAWINGS

The specific features, aspects and advantages of the present invention will become better understood with regard to the following description and accompanying drawings where:

FIG. 1 is a schematic block diagram of a computing device suitable for use in accordance with an embodiment of the present invention.

FIG. 2 is a schematic block diagram of a network environment suitable for use in accordance with an embodiment of the present invention.

FIG. 3 is a schematic block diagram of a database for use with a work management system in accordance with an embodiment of the present invention.

FIGS. 4A-4E illustrate user interfaces for specifying shift information in accordance with an embodiment of the present invention.

FIG. 5 illustrates a user interface for reviewing work reporting information in accordance with an embodiment of the present invention.

FIG. 6 is a process flow diagram of a method for reporting work information using telephony in accordance with an embodiment of the present invention.

FIG. 7 is a process flow diagram of a method for replicating shifts and corresponding tasks in accordance with an embodiment of the present invention.

FIG. 8 is a method for assigning workers to patients in accordance with an embodiment of the present invention.

FIG. 9 is a process flow diagram of a method for scheduling shifts having special classification activities (SCA) in accordance with an embodiment of the present invention.

FIG. 10 is a process flow diagram of a method for receiving reports on tasks and SCA in accordance with an embodiment of the present invention.

FIG. 11 is a process flow diagram of a method for calculating payable amounts for a shift including SCA in accordance with an embodiment of the present invention.

FIG. 12 is a process flow diagram of a method for generating alerts for SCA in accordance with an embodiment of the present invention.

FIG. 13 is a process flow diagram of an alternative method for generating alerts for SCA in accordance with an embodiment of the present invention.

FIGS. 14A-14B are process flow diagrams of methods for automatically scheduling providers for shifts in accordance with an embodiment of the present invention.

FIG. 15 is a schematic diagram of a family portal in accordance with an embodiment of the present invention.

FIG. 16 is a schematic diagram of a care log event for display in a family portal in accordance with an embodiment of the present invention.

FIG. 17 is a process flow diagram of a method for assembling a feed for a family portal in accordance with an embodiment of the present invention.

FIG. 18 is a process flow diagram of a method for handling interaction with a care log event in accordance with an embodiment of the present invention.

FIG. 19 is a process flow diagram of a method for handling prescriptions using a family portal in accordance with an embodiment of the present invention.

FIG. 20 is a process flow diagram of a method for receiving calendar events through a family portal in accordance with an embodiment of the present invention.

### DETAILED DESCRIPTION OF THE INVENTION

In the following description of embodiments of the present invention, reference is made to the accompanying drawings, which form a part hereof, and in which is shown by way of illustration specific embodiments in which the invention is may be practiced. It is understood that other embodiments may be utilized and structural changes may be made without departing from the scope of the present invention.

In the following description, numerous specific details are set forth in order to provide a thorough understanding of the present invention. However, it will be apparent to one skilled in the art that the present invention can be practiced without these specific details. In other instances, well known circuits, components, algorithms, and processes have not been shown in detail or have been illustrated in schematic or block diagram form in order not to obscure the present invention in unnecessary detail. Additionally, for the most part, details concerning networks, interfaces, computing systems, and the like have been omitted inasmuch as such details are not considered necessary to obtain a complete understanding of the present invention and are considered to be within the understanding of persons of ordinary skill in the relevant art. It is further noted that, where feasible, all functions described herein may be performed in either hardware, software, firmware, digital components, or analog components or a combination thereof, unless indicated otherwise. Certain terms are used throughout the following description and Claims to refer to particular system components. As one skilled in the art will appreciate, components may be referred to by different names. This document does not intend to distinguish between components that differ in name, but not function. In the following discussion and in the claims, the terms “including” and “comprising” are used in an open-ended fashion, and thus should be interpreted to mean “including, but not limited to . . . ”

Embodiments of the present invention are described herein. Those of ordinary skill in the art will realize that the following detailed description of the present invention is illustrative only and is not intended to be in any way limiting. Other embodiments of the present invention will readily suggest themselves to such skilled persons having the benefit of this disclosure. Reference will be made in detail to implementations of the present invention as illustrated in the accompanying drawings. The same reference indicators will be used throughout the drawings and the following detailed description to refer to the same or like parts.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with applications and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

The following description relates to scheduling systems for use in managing work taking place at various remote premises. The scheduling system finds particular application for home health-care scheduling and monitoring. The scheduling system may be useful for scheduling multiple daily work shifts of home care providers wherein information on certain measured outcomes is requested of the home care providers. In one embodiment, the system includes a scheduling system configured to organize work shifts of remote operating home care workers.

Work assignments may be organized into shifts associated with one or more workers and one or more recipients. Tasks are associated with the shift and have a status associated therewith that can be updated by a worker. Shifts may be replicated by cutting and pasting or by specifying a recurrence definition. The tasks associated with a shift may then be replicated according to the cut-and-paste operation or recurrence definition.

The status of tasks may be updated using a voice telephony system and/or by a computer interface. Using a voice telephony system, workers at a patient premise call from a phone on a recipient premise and report completion, non-completion and/or status of tasks. Voice comments for non-completed tasks or to report other information may also be recorded and stored by a work management system. Managers, clients, and concerned parties may view shift records and view the status of tasks as well as review any voice comments. The status of tasks may also be reported from a computer located on the recipient premise and text comments may be viewable by managers, clients, and concerned parties from a web portal accessible using a computing device, such as a tablet computer.

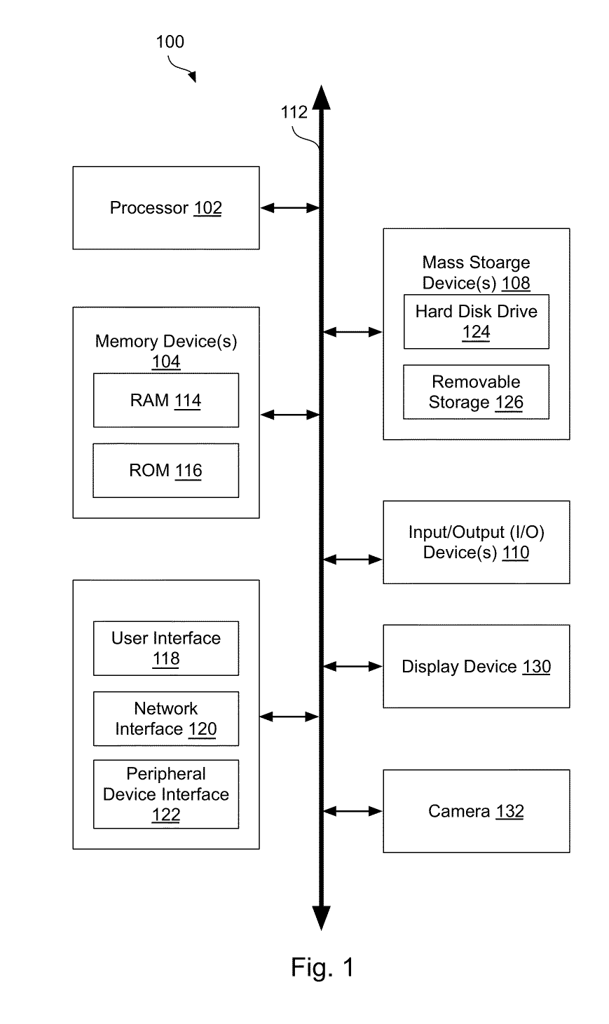

FIG. 1 is a block diagram illustrating an example computing device 100. Computing device 100 may be used to perform various procedures, such as those discussed herein. Computing device 100 can function as a server, a client, or any other computing entity. Computing device can perform various monitoring functions as discussed herein, and can execute one or more application programs, such as the application programs described herein. Computing device 100 can be any of a wide variety of computing devices, such as a desktop computer, a notebook computer, a server computer, a handheld computer, tablet computer and the like.

Computing device 100 includes one or more processor(s) 102, one or more memory device(s) 104, one or more interface(s) 106, one or more mass storage device(s) 108, one or more Input/Output (I/O) device(s) 110, and a display device 130 all of which are coupled to a bus 112. Processor(s) 102 include one or more processors or controllers that execute instructions stored in memory device(s) 104 and/or mass storage device(s) 108. Processor(s) 102 may also include various types of computer-readable media, such as cache memory.

Memory device(s) 104 include various computer-readable media, such as volatile memory (e.g., random access memory (RAM) 114) and/or nonvolatile memory (e.g., read-only memory (ROM) 116). Memory device(s) 104 may also include rewritable ROM, such as Flash memory.

Mass storage device(s) 108 include various computer readable media, such as magnetic tapes, magnetic disks, optical disks, solid state memory (e.g., Flash memory), and so forth. As shown in FIG. 1, a particular mass storage device is a hard disk drive 124. Various drives may also be included in mass storage device(s) 108 to enable reading from and/or writing to the various computer readable media. Mass storage device(s) 108 include removable media 126 and/or non-removable media.

I/O device(s) 110 include various devices that allow data and/or other information to be input to or retrieved from computing device 100. Example I/O device(s) 110 include cursor control devices, keyboards, keypads, microphones, monitors or other display devices, speakers, printers, network interface cards, modems, lenses, CCDs or other image capture devices, and the like.

Display device 130 includes any type of device capable of displaying information to one or more users of computing device 100. Examples of display device 130 include a monitor, display terminal, video projection device, and the like. The computing device 130 may additionally include a digital camera 132, scanner, or other image input device operably coupled thereto.

Interface(s) 106 include various interfaces that allow computing device 100 to interact with other systems, devices, or computing environments. Example interface(s) 106 include any number of different network interfaces 120, such as interfaces to local area networks (LANs), wide area networks (WANs), wireless networks, and the Internet. Other interfaces include user interface 118 and peripheral device interface 122.

Bus 112 allows processor(s) 102, memory device(s) 104, interface(s) 106, mass storage device(s) 108, and I/O device(s) 110 to communicate with one another, as well as other devices or components coupled to bus 112. Bus 112 represents one or more of several types of bus structures, such as a system bus, PCI bus, IEEE 1394 bus, USB bus, and so forth.

For purposes of illustration, programs and other executable program components are shown herein as discrete blocks, although it is understood that such programs and components may reside at various times in different storage components of computing device 100, and are executed by processor(s) 102. Alternatively, the systems and procedures described herein can be implemented in hardware, or a combination of hardware, software, and/or firmware. For example, one or more application specific integrated circuits (ASICs) can be programmed to carry out one or more of the systems and procedures described herein.

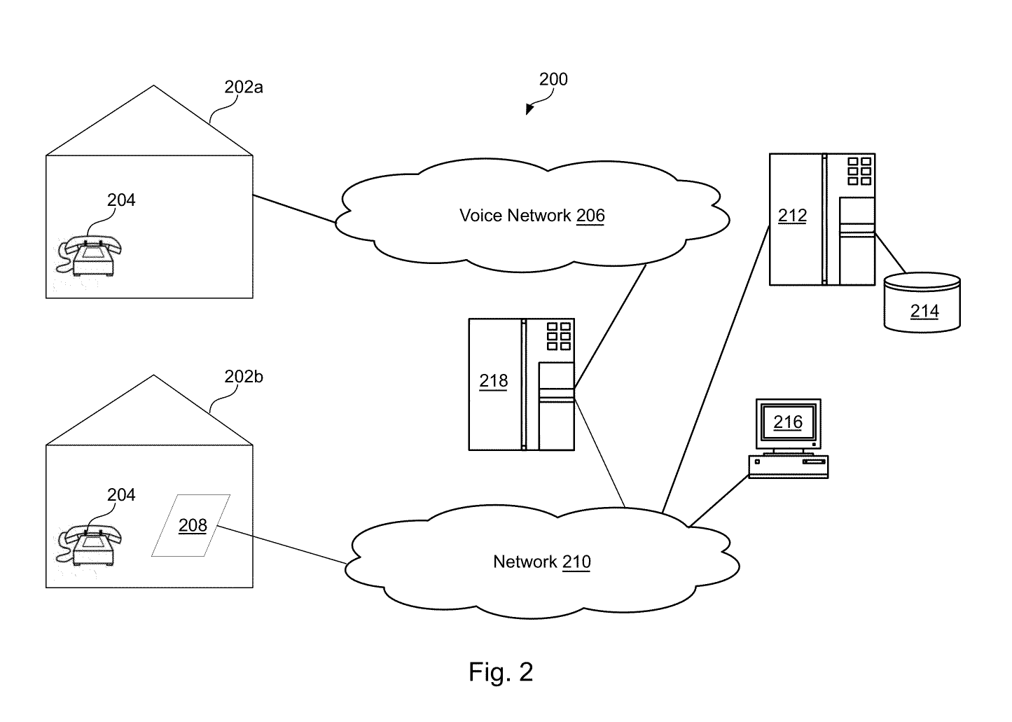

FIG. 2 is a block diagram of an example network environment suitable for use in accordance with one or more embodiments of the present invention. Work may be performed by employees in various premises 202a-202b, which may be client owned or recipient owned premises 202a-202b. The premises 202a-202b may have one or more telephones 204 installed therein that are connected to a voice network 206. The voice network may be a POTS telephone network, cellular network, voice-over-IP (VOIP), network or any other network suitable for transmitting and receiving analog or digital voice information.

The premises 202a-202b may have one or more computing devices 208 provided by a provider of work or a client or recipient of work. The computing device 208 may be a tablet computer, smart phone, computer terminal, or other computing device. The computing device 208 may have some or all of the attributes of the computing device 100. The computing device 208 may connect to a network 210 by means of a wired or wireless connection. The network 210 may include the Internet and one or more intermediate local area networks (LAN).

A work management server 212 may also connect to the network 210 directly or by means of one or more intervening LANs. The work management server 212 may have some or all of the attributes of the computing device 100. The work management server 212 may facilitate the assignment and monitoring of work taking place at a plurality premises 202a-202b remote from the work management server 212. The work management server 212 may host or be operably connected by an intervening network to a database 214 containing information regarding work scheduled to take place at the remote premises 202a-202b. The work management server may receive information regarding activities taking place on the remote premises from a computing device 208 or telephone 204 located at the remote premises 202a-202b.

Telephones 204 may communicate with the work management server 212 by means of a voice server 218 operable to route telephone network traffic over a digital network. The voice server 218 may be operable to convert voice information input to the telephone 204 to text and/or convert voice messages to digital files that may be routed over the network 210. Likewise, the server 218 may be operable to convert text received into voice messages routed over the voice network 206. The server 218 may be provided by a commercial venture such as Twilio which provides application programming interfaces (APIs) which are readily usable by those skilled in the art of software programming to build computer-enabled applications which use telephony, including voice recognition, voice-to-text automated transcription, text-to-voice technologies, and text messaging, to serve a variety of purposes.

One or more of workers, managers, clients, and recipients of work, and other concerned parties may access information regarding activities taking place at the premises 202a, 202b by means of a workstation 216 in data communication with the network 210. The workstation 216 may be embodied as a computer, smart phone, tablet computer, or the like. The workstation 216 may have some or all of the attributes of the computing device 100.

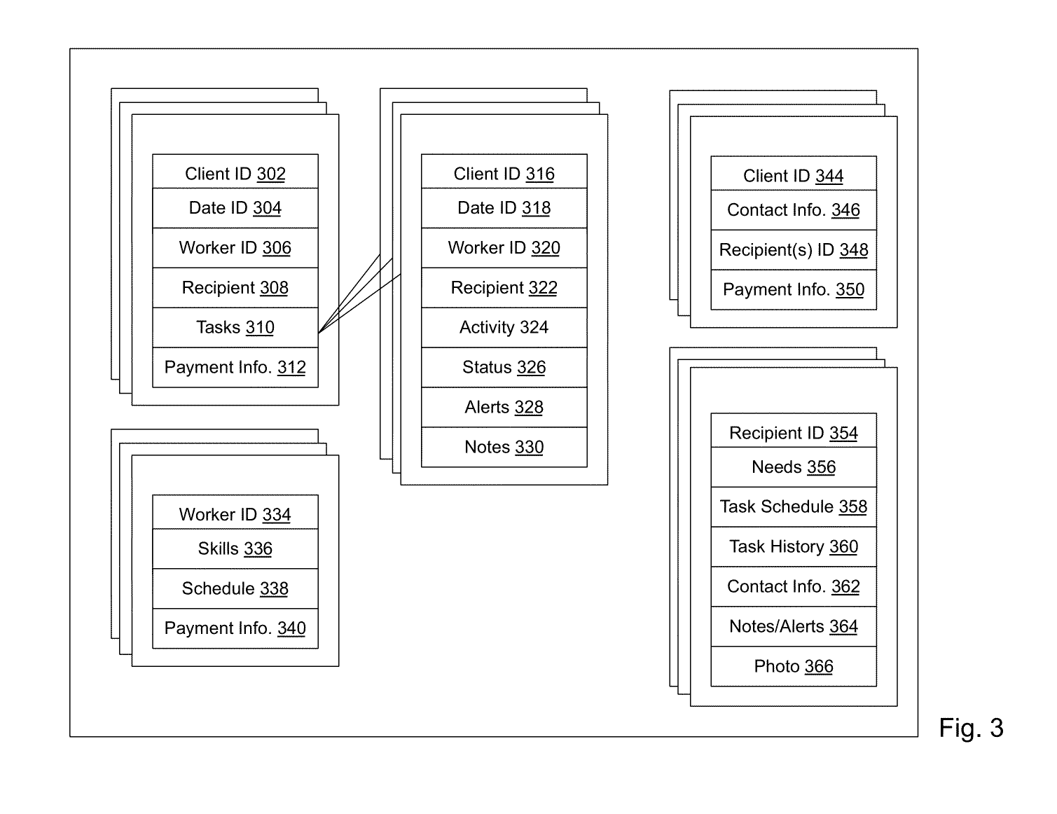

FIG. 3 illustrates a database 214 suitable for use in accordance with one or more embodiments of the present invention. The database 214 may store various records for managing and reporting on activities provided by workers on behalf of various recipients and clients. In one contemplated application, workers are home health care workers and the recipients are recipients of health care treatments and other assistance.

The database 214 may store shift records 300. A shift record 300 may define a workers activity for all or a portion of a day's work. A shift record 300 may also define work provided in a time interval on behalf of a single recipient. The shift record 300 may include a client identifier field 302 identifying the person or entity that is contracting for the provided work. The shift record 300 may additionally include a date field 304 defining a date and/or time interval during which work is to be performed. The shift record 300 may also include a worker identifier field 306 and a recipient identifier field 308.

A shift record 300 may have various tasks 310 associated therewith, either stored as part of the shift record 300 or stored as separate task records. The shift record 300 may also store payment information 312 regarding the shift, such as an amount owed to a worker and the amount owed by the client.

The database 214 may store task records 314. The task records 314 may inherit data from the shift record with which they are associated. Alternatively, the task records 314 may be linked to a corresponding shift record 300 and access shift information in this manner. Accordingly, the task record 314 may store or link to a client identifier 316, date and/or time identifier 318, worker identifier 320, and recipient identifier 322.

A task record 314 may additionally store a description 324 of the activity to be performed, the status 326 (e.g. completion status) of the task, any alerts 328 noted for the task, and any other notes or comments 330 recorded by a worker, recipient, manager, or client with respect to the task.

The database 214 may additionally store worker records 332 storing information about individual workers. A worker record 332 may include a worker identifier 334 (e.g., name or ID number), skills and abilities description 336, shift schedule 338, and payment information 340. The payment information 350 may contain information about shifts works and payments distributed to the worker. The skills and abilities description 336 may additionally include certifications possessed by a worker and dates they were obtained and/or dates on which the certifications should be renewed.

The database 214 may store client records 342 for clients contracting for services by the entity hosting the database 214. A client record may include a client identifier 344, contract information 346, identifiers 348 of recipients associated with the client, and payment information regarding services performed and payments received with respect to the client.

The database 214 may store recipient records 352 for individual recipients. A recipient record 352 may include a recipient identifier 354 (e.g., name or ID number), care needs 356 of the recipient, a task schedule 358 (and/or shift schedule) describing tasks scheduled on behalf of the recipient, a task history 360 and/or shift history recording tasks performed on behalf of the recipient, and contact information 362 for one or more of the recipient, concerned individuals, or relatives. The recipient record 352 may additionally record notes and/or alerts 364 with respect to the patient input by workers, concerned individuals, relatives, clients, doctors, and the like. The recipient record 352 may also include one or more photos 366 of the recipient and any other files that might be desired to include with the recipients profile.

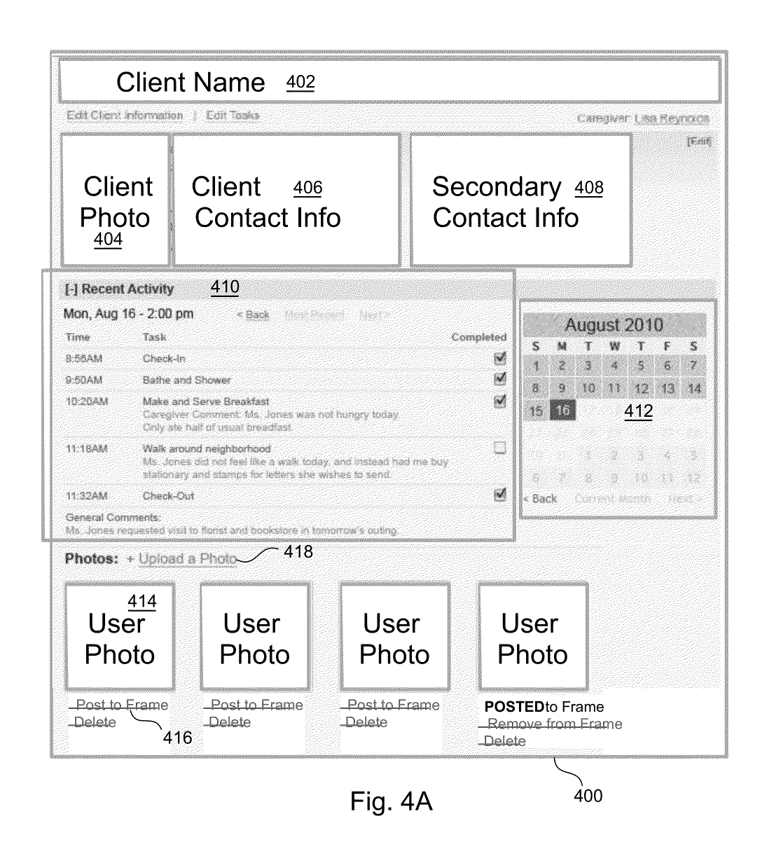

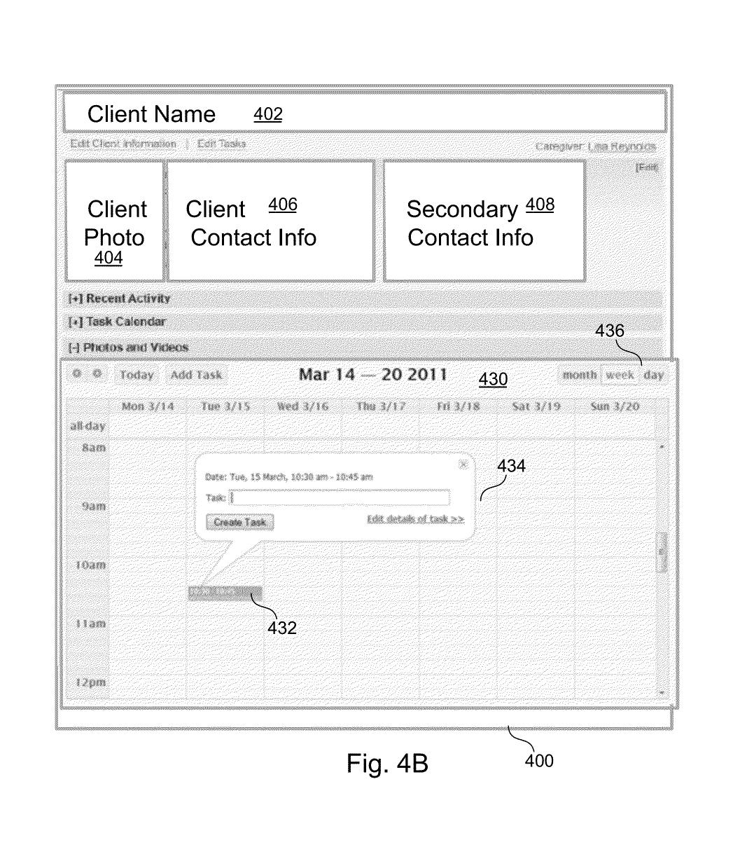

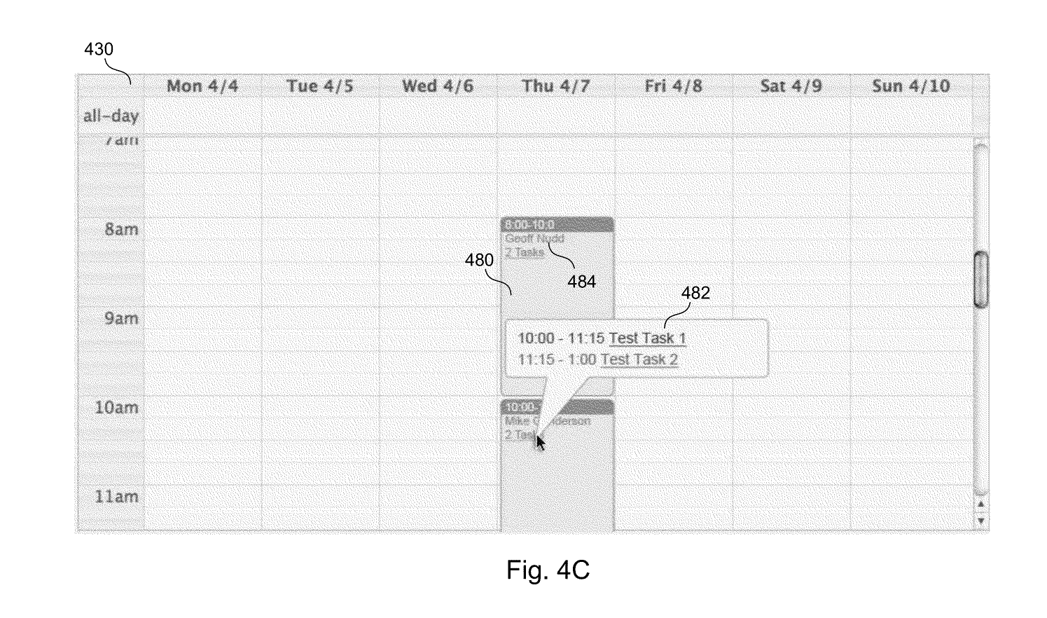

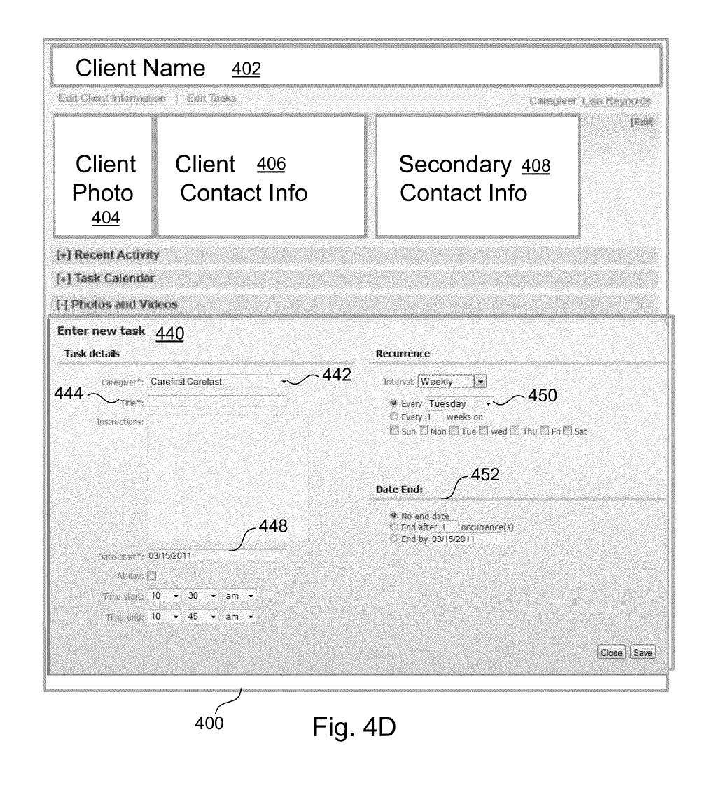

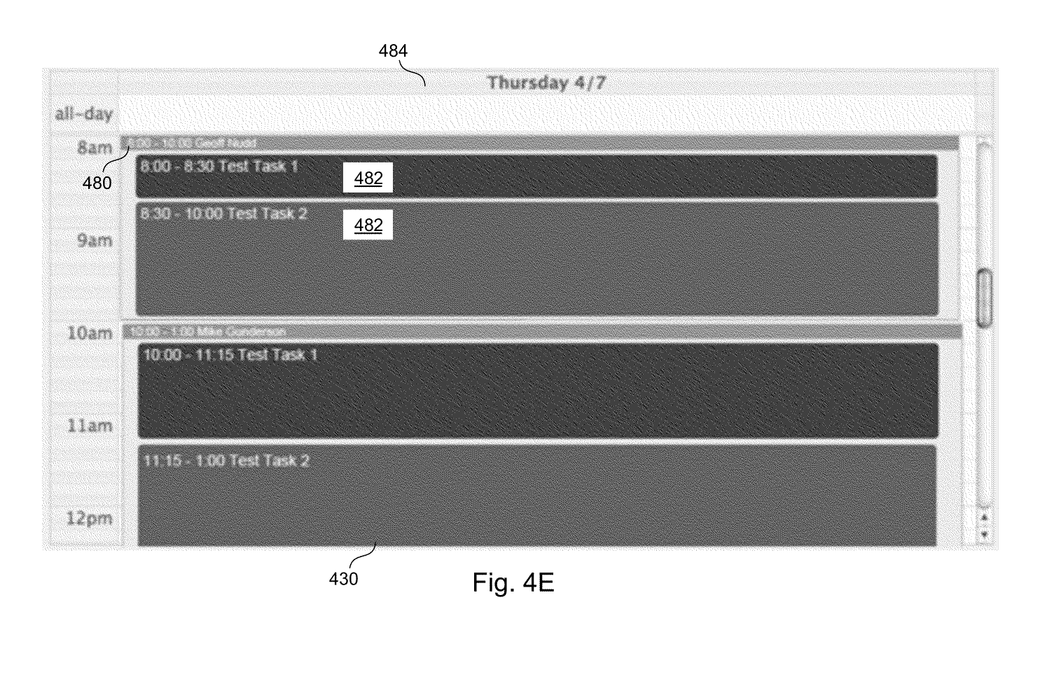

FIG. 4A is a wireframe diagram 400 that illustrates an interface of a web-based portal for a work management system which provides tracking and management of work, a photo storage service which enables the automatic display of photos which are uploaded via said web-based portal to a digital picture frame, the creation and management of non-event tasks and/or shifts, and the updating of the status of non-event tasks and/or shifts via a checklist interface accessible by a computer connectable to the Internet, a mobile tablet connectable to the Internet, and/or telephony.

In some embodiments, a touch screen tablet functioning as a digital picture frame and connected to the Internet, such as an Apple iPad, functions as a device by which one or more work providers manages and documents tasks and/or shifts at the client point-of-service. Element 402 illustrates a field by which identifying information of a client is displayed. Element 404 illustrates a field by which a photo of the client is displayed. Elements 406 and 408 illustrate fields by which contact information of the client is displayed. A variety of other user or user group profile information may also be displayed.

Elements 410 and 412 are interface elements enabling (1) tracking the completion and status of non-event tasks and/or shifts, (2) enabling work providers to provide input to said work management system via a separate interface (see FIGS. 4B and 4C) and/or via telephony as will be described, and/or (3) enable the client or family or other concerned individuals of the client to view tasks and/or shifts which have been completed by a work provider. In some embodiments, any user of the web portal 400 must be authenticated before being able to view the web portal 400 in order to protect the confidential and private information of the client. Means of authentication are well-known to those skilled in the art and include but are not limited to password protection and/or use of a personal identification number (PIN).

Element 410 illustrates a list of non-event tasks and/or the start time and/or end time of a shift (“checklist”). In some embodiments, the list provides status information for each task and/or shift which may include but is not limited to a variety of states such as to-be-completed, complete, incomplete, or exception. As shown in the present example, the task list 410 includes a variety of information for each task, including but not limited to the time at which a work provider completed a task and/or made an input relative to the task, a description of the task, comments submitted by the work provider, and whether or not the task was completed.

Element 412 illustrates a calendar input interface, which, when a day is clicked, queries the set of non-event tasks and/or shifts related to that day, including expected and/or actual start and end times for a shift, completed and incomplete tasks, and tasks which are planned in the future, and in a some embodiments displays said tasks in a task list 410.

In some embodiments, a work provider logs-in to the system from the point-of-service of the client to indicate the start time of the shift, is shown the non-event tasks which are to be completed on a computer 208, and marks tasks as complete and/or incomplete and/or enters comments as the work provider works towards the completion of tasks. In some embodiments, said comments and completion inputs from the work provider are transmitted via the network 210 to the work management server 212, and the completion information about the tasks and/or shift and the comments are shown in element 410, when one of a variety of authorized users, such as a manager or administrator, the work provider, the client, the recipient, or the family or colleagues of the recipient view the web portal 400. The above described functions and features advantageously achieve multiple benefits including transparency of work performed to the aforementioned parties.

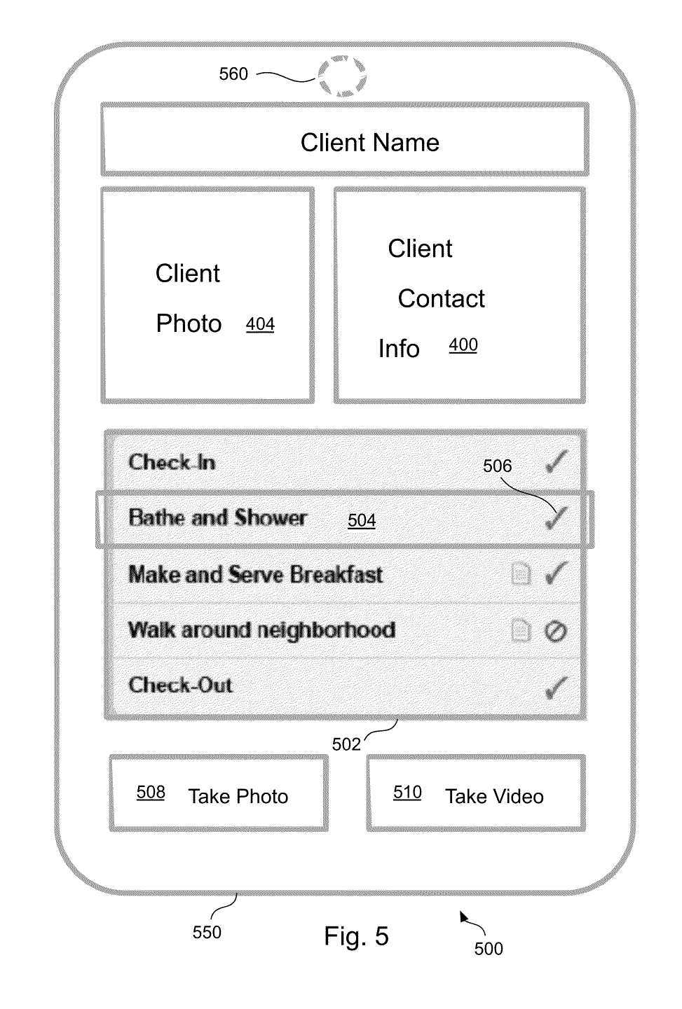

Element 418 illustrates a link to “Upload a Photo” which directs to a web-enabled interface which features an input field, a “Browse” button to find photo files on a local system, and an “Upload” button. Via these buttons and associated features, a photo file may be selected and uploaded to the work management system and thereby displayed in element 414 and stored. Systems and methods for uploading a photo file over the Internet are well known to those skilled in the art. The photo 414 may thereby be subsequently displayed by the system serving as a point of service input device for work providers, which thus in some embodiments serves as a digital picture frame. Via this interface, family members, friends, or other persons authorized by the client and/or work provider are able to both monitor work and upload photos 414 for display on the digital picture frame, which displays the photos 414 when said frame is not in use by the work provider for the provision and tracking of work (see FIG. 5). Element 416 is a pair of interface elements or hyperlinks to “Post to Frame” or “Delete”, which respectively trigger functions to designate the photo 414 for download by the digital picture frame, or to delete the photo 414 from the work management system. The links illustrated in element 416 are displayed when a photo 414 is displayed on the work management system, but which have not been designated for download by the digital picture frame.

Element 420 illustrates the text, hyperlinks and features which are in some embodiments displayed and enabled, respectively, when a photo 414 has been designated for display in the digital picture frame. The words “POSTED to Frame” indicate that the photo 414 has been designated for display in the digital picture frame. The “Remove from Frame” hyperlink enables the user to remove the designation that the photo 414 is to be displayed in the digital picture frame. The “Delete” hyperlink in element 416 enables the user to delete the photo 414 entirely from the work management system, and thereby to also delete the photo 414 from the digital picture frame.

FIG. 4B is a wireframe diagram that illustrates the task and/or shift input calendar interface 430 of a web-based portal 400 for a work management system which provides tracking, management and assignment of work, a photo storage service which enables the automatic display of photos which are uploaded via said web-based portal to a digital picture frame, the creation and management of non-event tasks and/or shifts, and updating of the status of non-event tasks and/or shifts via a checklist interface and/or telephony.

In some embodiments, the task and/or shift input calendar interface 430 is readily accessible and adjacent to the client-specific interface with elements 402, 404, 406 and/or 408, and/or the interface related to task and/or shift status 410, and/or the interface related to digital photo functionality containing elements 414, 416, 418 and/or 420. In task and/or shift input calendar interface 430, a user may create a new work task and/or shift by clicking any time on the calendar and/or by clicking an “Add Task” or an “Add Shift” button. In some embodiments, if the user clicks on a blank space on the calendar, the interface displays an “Add Shift” interface element, and if the user clicks on an area on the calendar in which there are shifts assigned, the interface displays an “Add Task” interface element wherein the task which is added will relate to said shift.

In some embodiment, the rapid addition of tasks is enabled by simply clicking on a time 432 in the calendar 430 in which there is a shift, typing the name and/or instructions of the Task, and clicking “return.” In another embodiment, the shift and tasks related to the shift may be made recurring by setting daily, weekly or monthly recurrence parameters for the shift, thus eliminating the need to separately set recurrence patterns for each individual task. This embodiment has the advantage of significantly saving time and improving accuracy relative to the prior art. In another embodiment of the present invention, the completion status of the non-event task and/or shift can be tracked via the interface described in element 410 based on inputs at the point-of-service from the work provider. If the task and/or shift has additional parameters including but not limited to detailed instructions or recurrence, the user may click “Edit details of the task” or “Edit details of the shift”, respectively, in the interface 434 to provide the additional parameters. In some embodiments, recurrence parameters are designated at the level of the shift. By way of example and without limitation, see FIG. 4E for an illustrative list of additional parameters that may be specified.

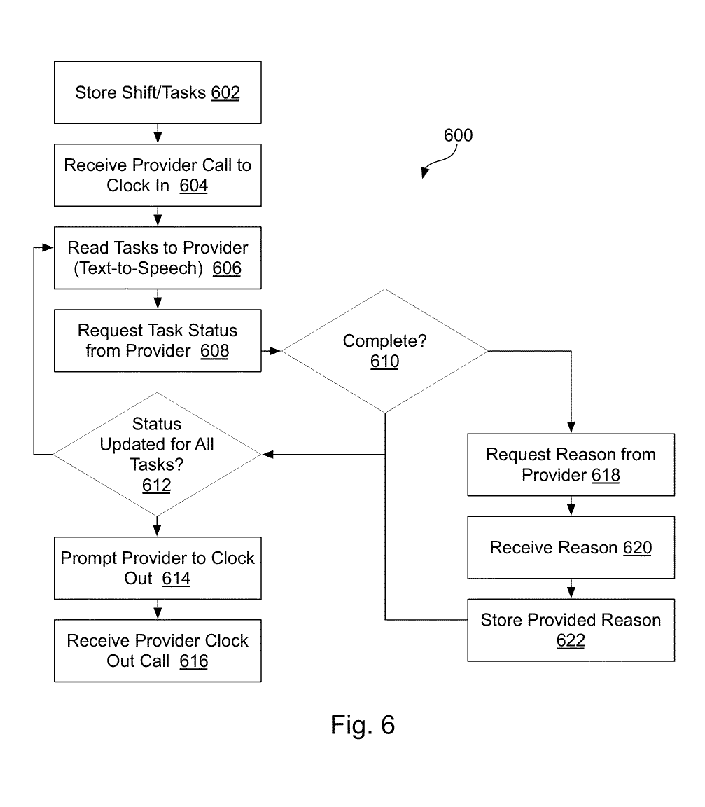

In some embodiments, tasks related to a shift inherit some or all of the properties of the shift including, by way of example and without limitation, the worker assigned, the recurrence, the pay rate to the worker, the billable rate to the beneficiary of the work, and/or other properties. In another embodiment, shifts and/or tasks may be queried from a database wherein the properties of said shifts and/or tasks may be used to calculate total and subtotal payables to a worker, total amounts to be billed to the beneficiary of work performed, and/or expenses. By way of example and without limitation, rates payable to the worker may be specified per shift, rates billable to the beneficiary of the work may be specified per shift, actual and/or planned hours to-be-worked may be recorded in conjunction with a telephony or web interface as described in FIGS. 5 and 6, and shifts and/or tasks may subsequently be queried and calculations performed of amounts payable and billable, respectively, as derived from hours and aforementioned rates for said shifts and/or tasks.

In some embodiments, various views of the calendar may be used by clicking inputs 436 including but not limited to a view of the current day another day, a week, or a month. As such, a level of calendar granularity convenient to the user may be viewed. In some embodiments, the calendar is implemented via Ajax, a group of interrelated web development methods used on the client-side to create interactive web applications.

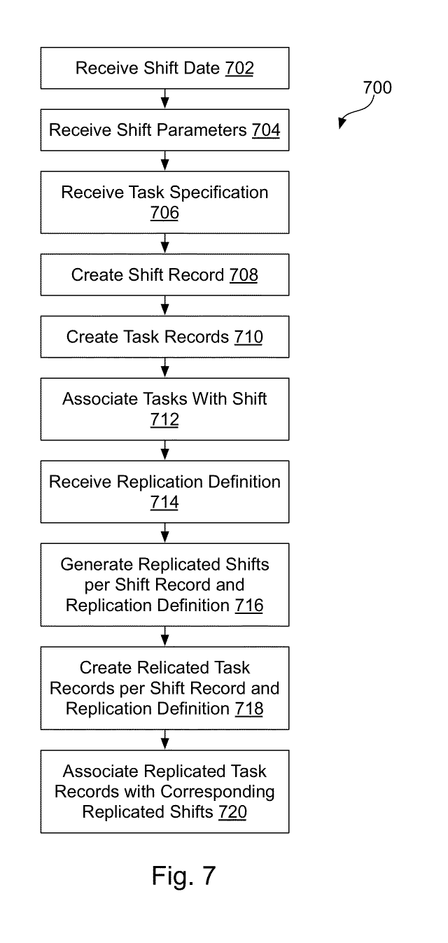

FIG. 4C is a wireframe diagram that illustrates the task and/or shift input calendar interface 430 of a web-based portal 400 for a work management system, with emphasis on the relationship between tasks 482 and shifts 480 and with a weekly calendar view. In some embodiments, each task 482 is related to a shift 480. Recurrence parameters of the shift 480 and/or task 482 may be set by the user such as illustrated with respect to FIG. 7. In some embodiments, one or more tasks 482 may be assigned to a shift 480, and recurrence may be set for the shift 480 wherein all tasks 482 related to the shift are similarly made to be recurring. Thus, the user need only specify all of the tasks 382 once for a given instance of a shift 480, and thus can in effect copy the tasks 482 to subsequent shifts 480 by defining recurrence. In some embodiments, one or more tasks 482 may be assigned to a shift 480, and the shift 480 and/or group of shifts within a time period, such as a day or week time period), may be copied and pasted to another time period. Thus, again the user need only specify all of the tasks 382 once for a given instance of a shift 480, and thus can in effect copy the tasks 482 to subsequent shifts by a cut-and-paste function. In some embodiments, properties 484 of the shift 480 are inheritable by the related tasks 482. By way of example and without limitation, the properties 484 of the shift 480 which are inheritable by the related tasks may include the worker, recurrence, location of work to-be-performed, beneficiary of the work, amounts payable to the worker, and/or amounts billable to the beneficiary of the work.

While the calendar interface 430 views shown in FIGS. 4A-4C are weekly, it may be anticipated by those skilled in the art that a monthly or daily view may be shown without materially altering the substance of the embodiments disclosed herein.

FIG. 4D is a wireframe diagram that illustrates a task and/or shift details portion 440 of a task and/or shift input calendar interface of a web-based portal 400 for a work management system. In some embodiments, the task and/or shift details interface 440 contains an input to assign the work provider 442. For illustrative purposes and by way of example, said input 442 may be to assign a work provider of the type “caregiver” wherein the work management system would be an in-home care work management system. The aforementioned is provided by way of example, and the invention has applicability to any variety of work types and work providers. In some embodiments, the name of the work provider is assigned a default value based on the primary work provider assigned to the particular client, but wherein another work provider may be designated specifically for the task and/or shift. In some embodiments, an input 444 captures the title and/or high-level instructions for the task and/or shift. In some embodiments, of the present invention, an input 446 enables input of detailed instructions for the task and/or shift. In some embodiments, an input 448 enables input of the start date, start time and end time of the task and/or shift and/or the designation of the task and/or shift as an “all day” task and/or shift. In some embodiments, the user may specify recurrence of the task and/or shift via a collection of inputs in interface areas 450 and 452.

The recurrence may be daily with a variety of parameters including but not limited to every day, every “x” number of days, every weekday, etc.; the recurrence may be weekly or every “y” weeks with a variety of parameters including but not limited to every week on one or more specific days of the week, or monthly on every “z” of every month, every “z” day (i.e. Thursday) of every month, etc. Systems for establishing recurrence for an event or meeting are well-known to those skilled in the art; however these systems for creating recurrence have not been applied to the creation of shifts with related tasks wherein the tasks may be specified only once relative to a given shift, and wherein subsequently the recurrence parameters of the shift may be set such that the related tasks are in effect copied with the shift. This approach advantageously significantly reduces time and improves accuracy in the specification of a set of two or more recurring tasks.

In some embodiments, the work management system includes the ability to specify recurring nonevent tasks and/or shifts in the calendar interfaces 430 and 440 wherein the completion status of the non-event tasks and/or shifts may be tracked via a checklist 310. Another aspect of some embodiments of the work management system is the ability to modify the status of a non-event task and/or shift remotely via a Internet-connected computer terminal as shown in FIG. 5, or via telephony as described in FIG. 6′.

FIG. 4E is a wireframe diagram that illustrates the task and/or shift input calendar interface 430 of a web-based portal 400 for a work management system. In some embodiments, each task 482 is related to a shift 480. Recurrence parameters of the shift 480 and/or task 482 may be set by the user as described hereinabove. In some embodiments, one or more tasks 482 may be assigned to a shift 480, and recurrence may be set for the shift 480 wherein all tasks 482 related to the shift are similarly made to be recurring. Thus, the user must only specify all of the tasks 482 once for a given instance of a shift 480, and thus can in effect copy the tasks 482 to subsequent shifts 480 by defining recurrence.

In some embodiments, properties of the shift 484 are inheritable by the related tasks 482. By way of example and without limitation, the properties of the shift 484 which are inheritable by the related tasks may include the worker assigned to the shift 484 and/or tasks 482, recurrence, location of work to-be-performed, beneficiary of the work, amounts payable to the worker, and/or amounts billable to the beneficiary of the work.

While the wireframes shown in FIGS. 4A-4E illustrate particular interface layouts for a work management system, one skilled in the art can anticipate many other specific variations which accomplish the features and benefits of the disclosed embodiments. Additionally, many of the elements such as 402, 404, 406, 408, 414, 416, 418, 420 and others may be generalized for use of the disclosed invention in the context of a social networking website, photo sharing website, or other system.

A specific case of the disclosed work management system is the application of said system for in-home care agencies wherein a multitude of clients receives in-home care and the work provider is a caregiver. The present work management system invention is particularly valuable in improving the lifestyle and happiness of elderly patients receiving in-home care from a caregiver by enabling the adult children and family of elderly patients to keep track of the provision of care and also to share said photos with the elderly patients. For an in-home care agency which manages care plans for a number of clients and which manages a number of caregivers, the system provides real-time transparency to care and a simple, easy-to-use interface for scheduling care.

FIG. 5 is a wireframe diagram that that illustrates an interface for caregivers and their patients or clients which are a specific instance of the aforementioned work management system described herein, and which may be displayed on an Internet connectable computer or touch screen tablet, e.g., Apple iPad, which is used by a caregiver to manage and document care tasks and/or shifts, and which also functions as a digital picture frame when not in use by the caregiver or other users. It may be appreciated by those skilled in the art that features described herein as accruing to the benefit of caregivers could be generalized to work providers of other types and accrue to the benefit of any number of varieties of remote work providers, and similarly the care management features and benefits described can accrue to the benefit of any variety of organizations involved in the management of remote work providers.

Element 500 illustrates a computer system with an interface 550, and shows an embodiment in which said computer system 500 is a touch screen computer tablet in which inputs to the computer system may be made by the user by touching the display screen interface 550, and which includes a built-in digital camera 560 which can take digital photographs that in turn can be stored, manipulated and transmitted by the computer system 500. Such computer systems 500 are well known and are widely distributed and sold, including by way of example the Apple iPad.

Element 500 illustrates the touch screen tablet in a mode in which the interface 550 is configured to be used by a caregiver as part of a work management system to manage and track the completion of care tasks and/or shifts. The interface 550 may be embodied as a web portal or other web interface. In some modes, the interface 550 is configured for the display of photos occupying most or all of a screen in accordance with the system's 500 additional capability as a digital picture frame. Element 502 is a list of tasks and/or shifts which are to be completed by the caregiver. In some embodiments, the caregiver may click or otherwise input to any individual task and/or shift 504 listed to changes its status, by way of example, from “Incomplete” to “Complete.” In some embodiments, the caregiver may double-click or otherwise input to any individual task and/or shift listed 504 to write one or more comments relative to the task and/or shift 504. In some embodiments, each task and/or shift 504 shows one or more completion indicators 506 which indicates the status of the task and/or shift 504, and/or one or more indications 506 that comments have been made about the task 504, and/or one or more indications 506 there are detailed notes about the task and/or shift 504 which may be stored on the work management system, and wherein the absence of such a displayed indicator 506 can also indicate the status of a task and/or shift 504. In some embodiments, the caregiver may input a specific measurement such as blood pressure or other medical reading in updating the status of the task and/or shift 504

A variety of information may be provided by the one or more indicators 506 for each task and/or shift 504. In some embodiments, after the caregiver changes the status of one or more tasks and/or shifts 504 on the list 502, the changes in the status of the one or more tasks and/or shift are transmitted to the work management system wherein the updated status of the tasks and/or shifts 504 can be displayed on the list of tasks and/or shifts 410 in the web portal interface illustrated in FIG. 4A.

Element 508 illustrates a button which is displayed on the interface 550 which when clicked, in some embodiments, configures the system 500 and camera 560 to take a digital photograph. In some embodiments, the caregiver authenticates his or her identity upon checking-in to a client site and/or prior to viewing tasks and/or shifts and/or changing the status of any tasks and/or shifts, such that said photo may be automatically uploaded to the work management system without subsequent authentication by the caregiver. In some embodiments, upon clicking the button 508, the caregiver is prompted by software running on the system 500 to confirm with a “yes” or “no” response whether or not the client has given explicit permission to the caregiver for such a photograph to be taken. In some embodiments, the caregiver is prompted via the interface to physically hand the system 500 to the client wherein the client is instructed to authenticate his or her identity with a password or other means in order to enable a photograph to be taken and uploaded to the work management system. The prompts described herein assist with compliance with laws that protect the privacy and confidential health information of clients.

In some embodiments, any photograph which is taken by the system 500 when used in conjunction with the work management system, for example, by clicking the button 508, is restricted such that it is not stored on the system 500 after the caregiver logs out of the work management system, and/or such that said photograph may only be stored permanently if it is transmitted over the Internet to the work management system, and/or stored on said work management system in a secure, remote database, wherein the photo is subsequently deleted from the device 500 after the caregiver logs-out of the present session with the device 500. Thus, photographs taken by the caregiver of the client are restricted in circulation such that the one or more photographs can only be viewed via secure work management interfaces such as illustrated in FIG. 4A.

Element 510 illustrates a button which is displayed on the interface 550 which when clicked, in some embodiments, configures the system 500 and camera 560 to take a digital video. The aforementioned functions and features for taking a photo by pressing the button 508 parallel those functions and features for taking a video by pressing the button 510, with the difference that the media file is a digital video file instead of a digital photo file in the case that button 510 is pressed.

The touch screen tablet computer system 500 may be operable in a mode in which the interface 550 is configured to display one or more photos in accordance with the system's 500 additional capability as a digital picture frame. This mode may be activated according to settings configured by the client, by the caregiver, by a caregiver administrator, or by other users and/or administrators of the integrated work management system, and/or may be preset in software stored on the system 500, or by other means 10 which are understood to those skilled in the art.

FIG. 6 is a flow diagram which illustrates the use of telephony instead of a touch screen tablet or other remote Internet interface as means to input updates to tasks and/or shifts in the work management system. In the aforementioned scenario in which the work management system is used for the management of an in-home care agency, there is sometimes the problem that the remote terminals by which task and/or shift information is updated are too expensive to be afforded by the client or by the in-home care agency. Moreover, many clients do not have Internet connectivity in their homes making it difficult and/or expensive to transmit updates of task status to the work management system. This problem, while acute in the in-home care agency industry, is also common to other industries which are dependent on a remote workforce that does not have readily available access to a computer terminal with connection means.

In the late 2000s, an increasing number of telephony services providers emerged such as Twilio (www.twilio.com) and Tropos (www.tropos.com) which provide application programming interfaces (APIs) which are readily usable by those skilled in the art of software programming to build computer-enabled applications which use telephony, including voice recognition, voice-to-text automated transcription, text-to-voice technologies, and text messaging, to serve a variety of purposes. Some embodiments disclosed herein solve this problem via the use of telephony and the new commercially available telephony services.

In some embodiments, the aforementioned calendaring systems may be interfaced with via a telephony system and/or via a computer connected to the Internet wherein the telephony system enables the work provider(s) assigned to a shift and/or non-event task to update the completion status or other properties of the shift and/or non-event task. In some embodiments, the computer-enabled system uses automated text-to-voice technology such as that enabled by commercial providers such as Twilio (www.twilio.com) accessible via an application programming interfaced (API) in conjunction with software code known by those skilled in the art to read instructions or other parameters of the shift and/or one or more non-event tasks to the person(s) assigned.

In some embodiments, the computer-enabled system accepts input via telephone from the person(s) assigned by which the person(s) updates the status of the shift and/or non-event task. By way of example, by pressing the number “one” on the telephone after the computer-enabled system reads the instructions and/or title for the non-event task, the person(s) assigned inputs a status update to mark the task as complete in the work management system. In some embodiments, if the person(s) assigned notes an exception to the expected status of the shift and/or non-event task such as updating the status as “incomplete,” then the person may communicate a voice message which is associated with the shift and/or task and/or group of tasks which communicates additional information which may include, by way of example, the reason that the shift and/or non-event task was not completed, or the reason at which the clock-in and/or clock-out time was not as expected.

In some embodiments, the voice message is stored in a system accessible via the Internet by which the status of one or more tasks and/or shifts (the “checklist”) may be viewed by one or more users. In some embodiments, a transcript of the voice message is recorded and displayed next to the relevant shift(s) and/or non-event task or group of tasks. In some embodiments, the transcript of the voice message is created via automated computer-enabled voice-to-text translation as enabled by commercial providers such as Twilio accessible via API in conjunction with other software code, the implementation of which is known to those skilled in the art. Alternatively, a recording of the voice message may be accessed by means of a link displayed adjacent to shift information.

By way of example and without limitation, the voice message or its transcription may be displayed in a checklist on a web portal 400 such as illustrated in element 410 of FIG. 4A. In some embodiments, the tasks and/or shifts are entered to the work management system via a calendar interface 430 in a web portal 400 with the additional features of being able to specify recurrence via, for example, inputs 450 and 452. In some embodiments, the tasks and/or shifts are entered relative to a specific client and the client contact information 406 includes the location at which the service is to be provided and the telephone number of the client.

The method 600 for managing work via telephony includes storing 602 shifts and tasks in a work management system using some or all of the methods described herein for entering a shift and tasks and generating replicated or recurrent shifts and tasks as described herein. A call from a work provider is received 604, such as from a phone 204 located on the premises 202a-202b of a work recipient. For example, the work provider dials-in from a designated phone number from the point-of-service in order to clock-in. The time the call is received 604 may be recorded by the work management system to indicate the worker's clock-in time for purposes of pay calculation. In some embodiments, the work management system compares the caller ID of the telephone from which the work provider is calling to the contact information 406 of the client to verify that the work provider is at the proper location.

Tasks are then converted from text to speech and read 606. Reading 606 the tasks from text to speech may be performed by one of or a combination of the work management server 212 and a voice server 218. In step 606, after clock-in to the shift, tasks may be read to the work provider sequentially using text-to-voice technology by passing text information related to the task such as the desired start time, the desired end time, the title or high-level instructions, and/or detailed instructions to a telephony service provider from the work management system via API. Telephony service providers such as Twilio (www.twilio.com) and related APIs are well-known to those skilled in the art. Thus, the work provider is prompted with the task(s) to be performed.

In some embodiments of the present invention, all of the tasks to be performed within a specific period of time or shift are automatically read to the work provider in the first reading 606 after the clock-in step 604 wherein there are no interruptions for prompts requesting completion status so that the work provider can be informed of the tasks to be performed. In subsequent readings, following the reading of each task there is a request 608 transmitted to the work provider to update the status of each individual task. In some embodiments, there is no such initial “read through” of tasks. Instead, after clock-in step 604, the tasks are read one at a time in step 606 and after each task is read, the work provider is prompted 608 to answer whether or not the task has been completed.

The work provider can respond to the prompts using means well-known to those skilled in the art such as by pressing a digit on the phone or responding verbally. The commercially available telephony service interprets the input from the work provider per rules specified in software code as is known to those skilled in the art. If the task if found 610 to have been reported as complete, then the method 600 may include evaluating 612 whether or not there are additional tasks for which status has not been updated. If not all tasks are updated, then the next task is read 606 and the status requested 608 and the method continues as shown.

If the status of all tasks is found 612 to have been updated, then the work provider may be prompted 61 to clock-out. If the work provider has no further work to do at the point-of-service, then clocking-out of the work provider is received 616. The clock-out time may be noted by the work management system and recorded, such as for pay calculation purposes. Clocking out may additionally include receiving a signature in the form of a personal identification number (PIN) or voice signature over the telephone network 206. In some embodiments, a voice signature may be a verbal recitation of a PIN or the work provider's name. A work provider may be also be prompted to verbally attest to completion of the tasks associated with the completed shift and the work provider's response may be stored as the voice signature. Alternatively, a PIN may be entered using a keypad. A voice signature may be stored for later reference. Additional details regarding signatures may be found in U.S. application Ser. No. 13/420,506, which is hereby incorporated herein by reference in its entirety.

If the worker is found 610 to have reported that a task has not been completed, the work provider may be prompted 618 to record a reason that the task was not completed, and a voice explanation may be received 620. The reason received 620 may be stored 622 by the work management system as a voice message via means well-known to those skilled in the art and enabled by telephony service providers such as Twilio. Alternatively or additionally the response may be automatically transcribed to text using voice-to-text technologies provided by telephony service providers such as Twilio. After receiving 620 the reason, processing may return to step 612 and processing continues as described above.

In some embodiments, a task checklist is accessible via web portal 400, preferably in a checklist interface 410. In some embodiments, the work management system compares the caller ID of the telephone from which the work provider is calling to the contact information 406 of the client to verify that the work provider is at the proper location during the point in time at which status for each task is updated. Alternatively, a GPS coordinate of a cell phone owned by an employer, worker, or recipient may be transmitted to the work management system and used to verify the worker's location. In some embodiments, the work provider can hang up the phone at any point and resume the process at the step at which the work provider last left-off by calling the telephony service phone number again.

In some embodiments as the status of tasks and/or shifts is updated via the telephony system, the updated status can be viewed via the web portal 400 via interface 410 as shown and described relative to FIG. 4. In some embodiments, alerts are provided via the web portal 400, via text messaging, via outbound calling as enabled via the telephony service, or other means known to those skilled in the art to the work manager, the work provider, persons associated with the client, or other stakeholders in the event that a clock-in is missed or if a task is not completed, completed, and/or marked with a status which is designated to trigger an alert. Thus, the telephony service in the work management system enables a variety of stakeholders to have real-time visibility of highly specific tasks without requiring a costly remote computer terminal such as, by way of example, a mobile computing tablet 500.

Considering now a specific application by way of example and without limitation to the aforementioned, an in-home care agency managing a multitude of patients or clients and a multitude of caregivers realizes a great number of benefits via usage of the aforementioned inventions. Today, many in-home care agencies use paper care journals at the point-of-care to manage care and record updates as to the completion of tasks. Unfortunately, the use of paper care journals makes it impossible for in-home care agency managers and family and adult children of elderly clients to closely observe the care provided.

The mobile tablet interfaces eliminate the need for paper care journals and enable real-time visibility to the point-of-care for in-home care agency managers and for the family of patients and clients. This significantly reduces costs and improves the quality of care. For situations in which a mobile interface cannot be afforded, the telephony service provides a low-cost means leveraging patients and/or client's existing phone systems to achieve the same benefits with a level of granular visibility to the care provided and tasks completed that did not previously exist. Additionally, the work management system disclosed provides an easy-to-use and intuitive means of scheduling a care plan via a calendar interface. Today, care plans and task scheduling are typically managed via paper care journals for in-home care agencies. When care plans are managed electronically, they are often managed with highly-detailed form templates that lack the dimension of scheduling of specific tasks at specific times. In the prior art, when a calendar is used, no greater granularity than a work shift is provided; the present solutions lack task-specific granularity as disclosed with regards to the present invention.

The shift and/or task input calendar interface disclosed provides very critical improvements to these systems by providing a robust, highly flexible means of scheduling very detailed care plans with associated times for each shift and/or task. Because of this critical enabling feature, it follows that the individual tasks can be output to a mobile tablet, an Internet connectable computer, and/or telephony services as described, and the status of tasks can also be updated via these channels. As such, it provides unprecedented visibility to the point-of-care enables new and beneficial features including but not limited to alerts if tasks that have been scheduled as part of the care plan are reported with an exception status.

FIG. 7 illustrates a method 700 for defining work schedules, particularly work schedules involving tasks performed at remote locations. The method 700 may be executed on a work management server 212 in response to inputs to a web interface presented on a user workstation 216 or some other device. Alternatively, the method 700 may be executed on a workstation 216 or other device and then corresponding changes may be made to a database 214 storing work management information.

The method 700 may include receiving 702 a shift date and receiving 704 other shift parameters. The other shift parameters may include a recipient of work, a provider of work, a work address, and other parameters defining the work to be provided. The specification of one or more tasks may also be received 706. A task includes a description of work to be performed. The task description may include one or both of a title and detailed description of the task. A task may or may not have a time associated therewith.

A corresponding shift record may be created 708 and one or more task records 710 may also be created. The task records may be associated 712 with the shift record. This may include one or both of including an identifier of the shift record in the task records or identifiers of the task records in the shift record. Associating 712 may additionally or alternatively include including the shift date in the task records 710. Associating 712 may also include recording one or more shift parameters of the shift with each of the one or more task records 710.

The method 700 may further include receiving 714 a repetition definition. Receiving 714 the replication definition may include cutting and pasting the shift in an interface, such as the interfaces disclosed herein and as known in the art of graphic user interfaces. The replication definition may define one or more different shift dates. Receiving 714 the replication definition may additionally or alternatively include receiving a recurrence definition defining recurrence of the shift. The recurrence definition may include an end date, a repetition interval, a day of the week and or month on which the shift is to be repeated, or any other time interval.

Replicated shifts may be generated 716 according to the replication definition. This may include generating 716 replicated shift records including the shift parameters as received 704 for the original shift and a replication date as defined according to the replication definition. For each replicated shift, one or more replicated task records may be created 718 by copying the tasks received 706 for the original shift, or by relating the tasks received 706 for the original shift to any shifts specified to occur thereafter via the receipt of specifications for recurrence 716. The replicated task records may be associated 720 with a replicated shift record such that each replicated shift record has associated therewith a copy of the original one or more tasks received 706 for the original shift.

Any of the replicated shifts or the replicated tasks associated therewith may be edited independently or as a group by changing the recurrence definition. Each of the replicated shifts may itself be replicated according to the method 700. Each of the shifts and the tasks associated therewith may be the subject of a status updating method, such as the method 600 described above with respect to FIG. 6.

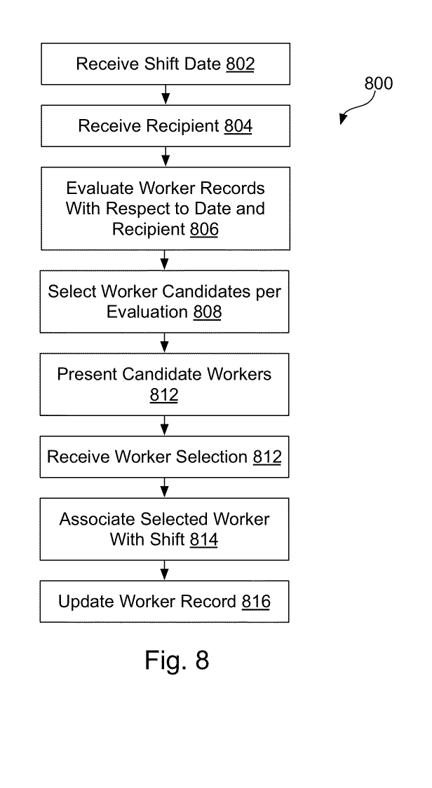

FIG. 8 illustrates a method 800 for assigning workers to shifts. The method 800 may be used to specify a shift, such as at step 702 of the method 700. The method 800 includes receiving 802 a shift data and receiving 804 a shift recipient, such as from a user input. The work management system evaluates 806 the shift date and the needs of the shift recipient with respect to availability and skills of workers. This may include evaluating the recipient records 352 and worker records 332 stored in the database 214. Worker candidates having availability on the shift date and skills corresponding to the recipient needs are then selected 808 according to the evaluation 806. The worker candidates may be transmitted and displayed 810, such in the web portal 400 displayed on a work station or tablet computer. A worker selection 812 may then be received and the selected worker associated 814 with the shift. The shift record may be created or updated to indicate the selected worker and the selected worker's record may be updated 816 to indicate the assignment. In some embodiments, selection 812 of a worker may be automatic, such as random or algorithmic selection from among the candidate workers. In such embodiments, presentation 810 of the candidate workers may be omitted.

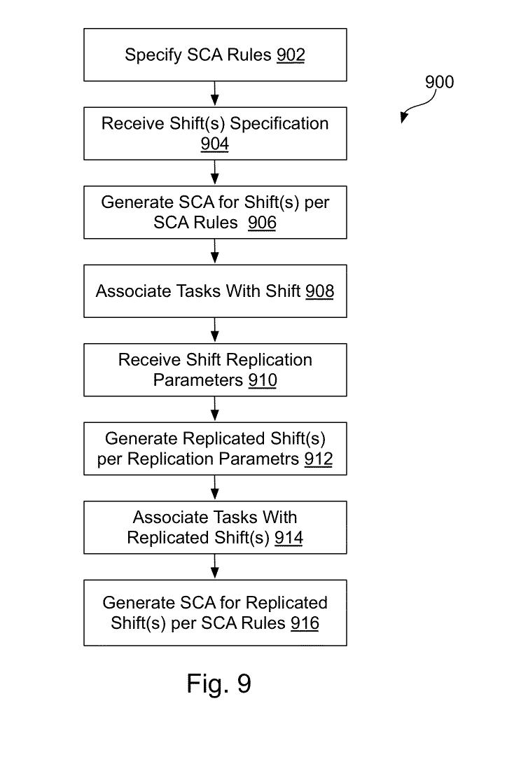

Referring to FIG. 9, in some instances certain activities other than tasks may need to be associated with a shift. For example, new regulations have required that the occurrence of personal activities such as meals, breaks, and sleeping are required to be reported and recorded for certain situations, such as 24-hour in-home care. The method 900 of FIG. 9 may be used to schedule these activities.

The method 900 may include specifying 902 “special classification activity” (SCA) rules. The SCA rules may define when certain activities must take place for a given shift, or for a given shift attributes. For example, the SCA rules may define rules for how many breaks must be taken per hours worked, how much sleep must occur per hours worked and/or for what time(s) of day, how many meals must be taken per hours worked and/or for what time(s) of day, or other activities. In addition to these types of rules, the number and distribution of such activities may be specified according to time of day. For example, the SCA rules may specify diurnal activities such as meals and breaks for daytime hours and nocturnal activities such as sleep for nighttime hours. Although the examples of SCA listed above are personal activities, any activity that may be required to be performed by a provider may be specified as an SCA or according to SCA rules.

One or more shift specifications may be received 904. The shift specification may include any of the parameters described hereinabove as appropriate for defining a shift. For example, the shift specification may include a date, start time and end time, a provider, a recipient, a supervisor, pay rate, and any other relevant parameters.

SCA may then be generated 906 for the shift according to the SCA rules and the shift specification. As noted above, SCA rules may specify activities that must occur according to a number of hours worked. Accordingly, a duration of the shift may be evaluated and SCA specified with a proper distribution in the shift according to comply with the SCA rules. As also noted above, SCA rules may specify activities according to a time of day. Accordingly generating 906 the SCA for the shift may include evaluating start and end times for the shift, determining the time of day, and selecting SCA according to the time of day and with the proper frequency according to the SCA rules. The SCA generated may be associated with the shift and may be processed and otherwise treated according to some or all of the functionality described hereinabove for tasks.

Tasks may also be associated with the shift as specified 904, such as according to some or all of the functionality for associating tasks with shifts as described hereinabove. The shift as specified and with any SCA or tasks associated therewith may be stored, such as by creating a shift record and associated task records for tasks and SCA as described hereinabove.

In some instances, an instruction to replicate a shift may be received 910 along with one or more shift replication parameters. For example, a shift may be replicated according to a recurrence definition or an instruction to copy, such as a cut-and-paste operation, as described hereinabove. In such instances, one or more replicated shifts may be generated 912 according to the shift replication parameters. For example, the shift replication parameters may specify a different date, start time, end time, provider, recipient, supervisor, or any other parameter.

Tasks associated with the original shift may be associated with the replicated shift, such as according to some or all of the functionality described hereinabove for associating tasks with shifts. SCA may be treated differently than tasks. In particular, rather than simply associating SCA from the original shift with the one or more replicated shifts, the replicated shifts, as defined according to the replicating parameters, may be analyzed according to SCA rules and SCA may be generated 916 for the replicated shifts according to the replicated shift attributes and the SCA rules. As already noted, replicated shifts may have a start time, end time, and date that may be used for applying SCA rules as described hereinabove. SCA as generated 916 may then be associated with the one or more replicated shifts.

As noted hereinabove, in some instances, actual replicated shifts may not be generated according to an instruction to replicate a shift. For example, where the instruction to replicate a shift defines a recurrence that may encompass many shifts, storage space may be conserved by saving the recurrence definition and refraining from generating actual shift records for each shift defined according to the recurrence definition. Shifts as defined per the recurrence definition may be generated and tasks associated therewith at some point prior to each shift, such as according to the method 900. In a like manner, SCA may be generated according to the methods described herein and associated with the replicated shifts just prior, e.g. one or two weeks prior, to the actual date of a particular replicated shift as defined according to the recurrence definition.

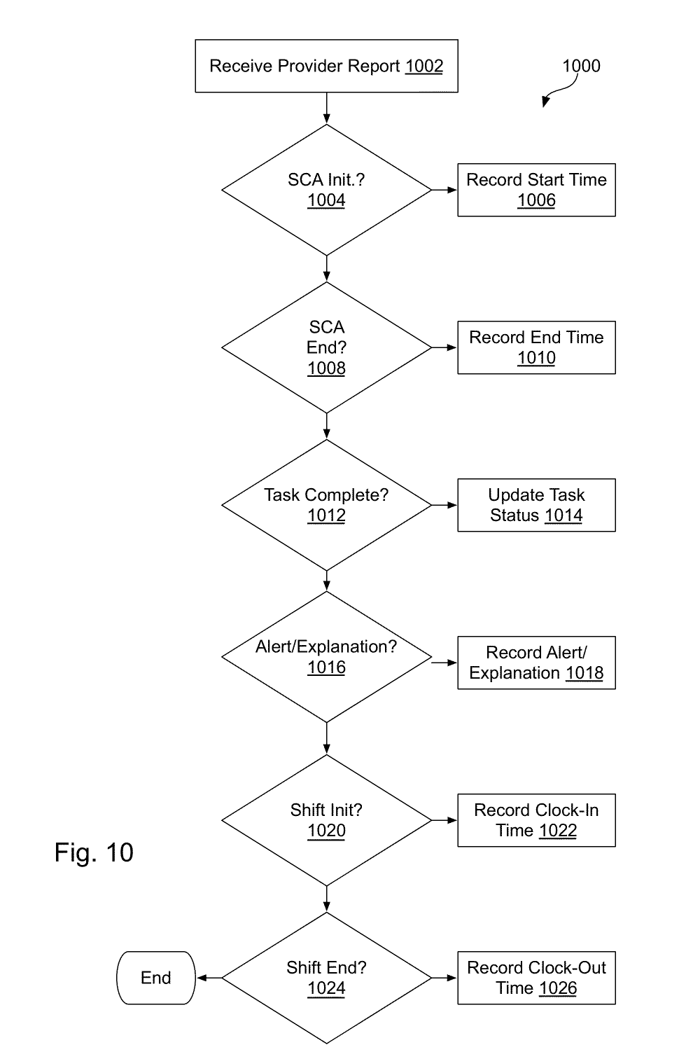

FIG. 10 illustrates a method 1000 for reporting the status of tasks and SCA. The method 1000 may be executed by a provider operating computer, tablet computer, smart phone or the like. The method 1000 may be executed using the telephone reporting methods described herein.

The method 1000 may include receiving 1002 a provider report. This may include receiving a key press in the context of the telephone reporting methods described hereinabove. Receiving 1002 may also include receiving a voice report that is subsequently converted to text. Receiving 1002 may also include receiving text message or a message from software executing on a device operated by a provider.

The report received may then be evaluated. If the report is found 1004 to be reporting the start of an SCA, then the start time may be recorded 1006, such as in a shift record for the shift the reporting provider is working or in a task record corresponding to the SCA. If the report is found 1008 to be reporting the stopping of an SCA, the stop time may be recorded 1010, such as in the same manner as the start time.

If the report is found 1012 to indicate that a task, such as a task other than an SCA, has been completed, then the status of that task may be updated 1014 such as by updating a shift record or task record corresponding to the completed task.

As noted above, various circumstances may occur where a provider is prompted or permitted to provide a comment or explanation. For example, where a task has not been completed or cannot be completed, a provider may be prompted to provide an explanation or otherwise given an opportunity to associate an explanation with a shift or task. Accordingly, if the report is found 1016 to be an alert or explanation from a provider, then the alert or explanation may be recorded 1018, such as by recording a voice message received over the phone or storing in association with a shift or task a textual message received by text message or from software operated by the provider.

As already discussed hereinabove, the provider may also clock in and clock out for a shift using a telephone system or using software operated by a provider. Accordingly, if the report is found 1020, 1024 to indicate the start of a shift or the end of a shift then the clock in or clock out time may be recorded 1022, 1026, respectively in association with the appropriate shift for the reporting provider.

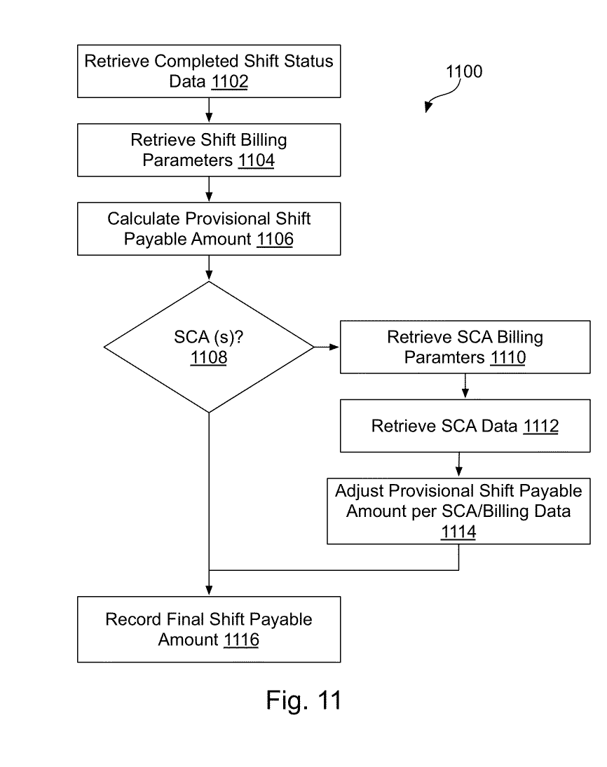

FIG. 11 illustrates a method 1100 for calculating a payable amount for a shift that includes both SCA and non-SCA tasks. An SCA may have a billing rate associated therewith that may be different from a billing rate associated with the shift or with the provider associated with a shift. In some instances, a provider may have an SCA billing rate associated therewith and a shift billing rate associated therewith. In any case, the time spent on a shift may be computed according to a shift billing rate and an SCA billing rate.

For example, the method 1100 may include retrieving 1102 shift status data for a completed shift. This may include retrieving 1102 a start time and end time for the shift and may include retrieving other data such as a date for purposes of computing any differential for holidays or night shifts. Billing parameters for the shift may also be retrieved 1104, this may include retrieving a shift billing rate associated with one or both of the completed shift and a provider that worked the completed shift. A provisional amount payable may be calculated 1106 according to the shift billing parameters. If the shift is found 1108 to include SCA, then SCA billing parameters may be retrieved 1110 and SCA data may also be retrieved 1112. The SCA billing parameters may include an SCA billing rate associated with one or both of the completed shift and the provider that worked the completed shift. The SCA data may include start times and end times for any SCA activity. The provisional shift payable amount may be adjusted 1114 according to the SCA data and SCA billing parameters. For example, if the hourly rate for SCA activities is different than that for the shift, then the amount due for a shift may be adjusted up or down accordingly. The final amount payable for a shift may then be recorded 1116 for use in making appropriate payments to the provider that worked the completed shift.

In some embodiments, rather than calculated a provisional amount payable, the hours worked based on the start time and end time of the shift may be reduced by the amount of time performing SCA before applying the billing rate for the shift. The SCA billing rate may then be applied to the amount of time spent on SCA to obtain a SCA payable amount. The shift payable amount and SCA payable amount may then be summed to obtain a final figure.

Any of these methods for computing a payable amount may also be used to calculate an amount owed by a person that is paying for the service associated with the completed shift by replacing the payable amount with an amount due and using appropriate billing rates for the shift and SCA.

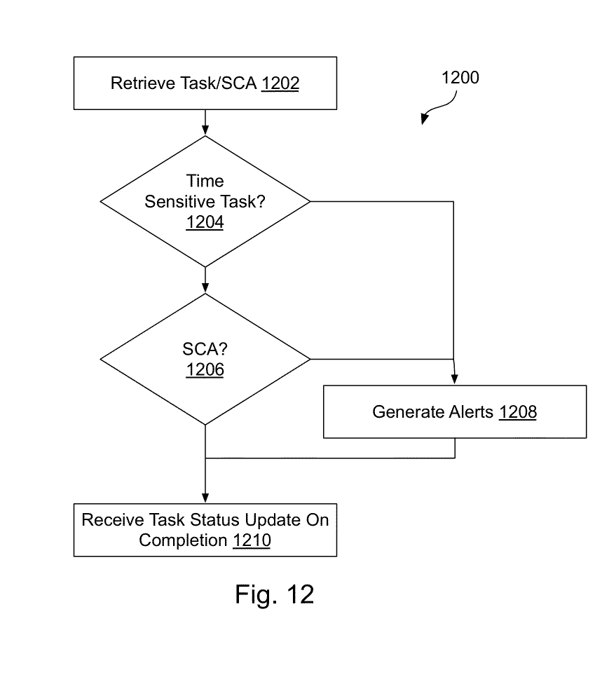

FIG. 12 illustrates a method 1200 for providing alerts. SCA and tasks may have alert parameters associated therewith. The alert parameters may define a time by which the SCA or task should be one or both of initiated and completed. The alert parameters may also specify who should receive reminders and alerts in connection with the task or SCA.

Accordingly the method 1200 may include retrieving 1202 a task or SCA, evaluating 1204 whether the retrieved activity is a time sensitive task and evaluating 1206 whether the activity is an SCA. If the activity is found to be either a time sensitive task or SCA, an alert may be generated 1208. Generating 1208 an alert may include making an entry or instruction that will prompt generation of an alert at an appropriate time. Generating 1208 an alert may include making an entry or instruction that will prompt evaluation of the status of the SCA or task at an appropriate time and generate an alert if the SCA or task has not been one of started or completed depending on the type of alert. In some embodiments, alerts may include reminders to a provider to start or end an SCA or task at an appropriate time. Alerts may include warnings or alerts provided to supervisors or concerned parties if a task or SCA is not started or completed within a proscribed time period. Upon completion or initiation of a task or SCA, status updates may be received 1210 according to methods described herein.

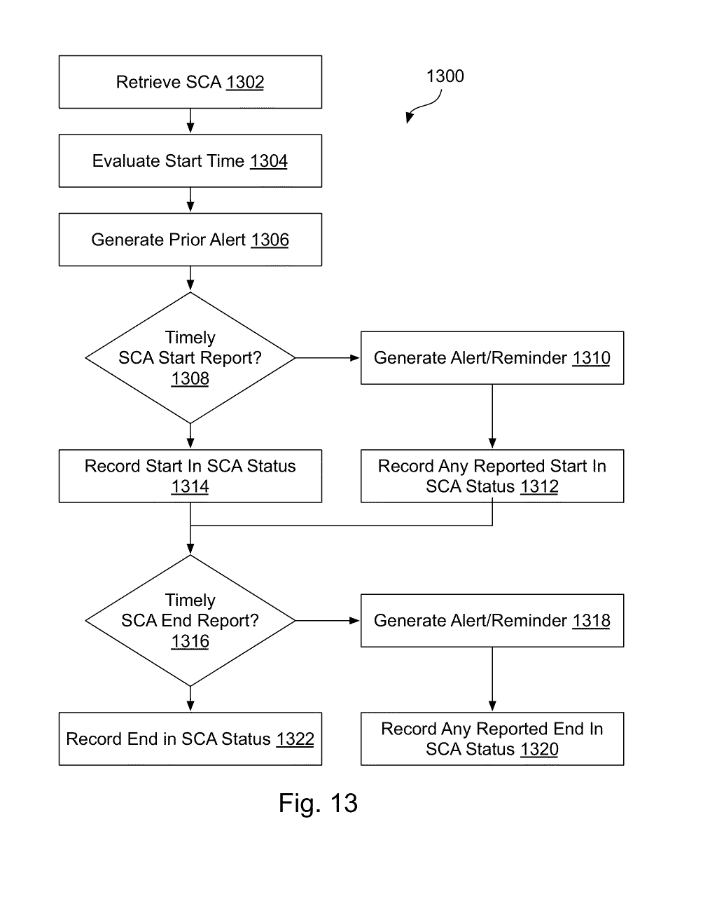

FIG. 13 illustrates an alternative method 1300 for providing alerts for an SCA. The method 1300 may include retrieving 1302 an SCA for a shift and evaluating 1304 a start time defined for the SCA. A prior alert may be generated 1306 according to the start time, such as N minutes before the start time.

The method 1300 may further include evaluating 1308 whether a timely report of starting the SCA has been received. If not, one or more alerts may be generated 1310 and sent to one or more of the provider for the shift, the provider's supervisor, and concerned individuals. Any reports of commencements of the SCA may be received and recorded 1312 following generation 1310 of the alert. If a timely report of starting the SCA is found 1308 to have been received, this start of the SCA may likewise be recorded 1314. This may include recording a start time for the SCA.

The method 1300 may further include evaluating 1316 whether a timely report of ending the SCA has been received. If not, one or more alerts may be generated 1318 and sent to one or more of the provider for the shift, the provider's supervisor, and concerned individuals. Any reports of ending of the SCA may be received and recorded 1320 following generation 1310 of the alert. If a timely report of ending of the SCA is found 1316 to have been received, this ending of the SCA may likewise be recorded 1322. This may include recording an end time for the SCA.

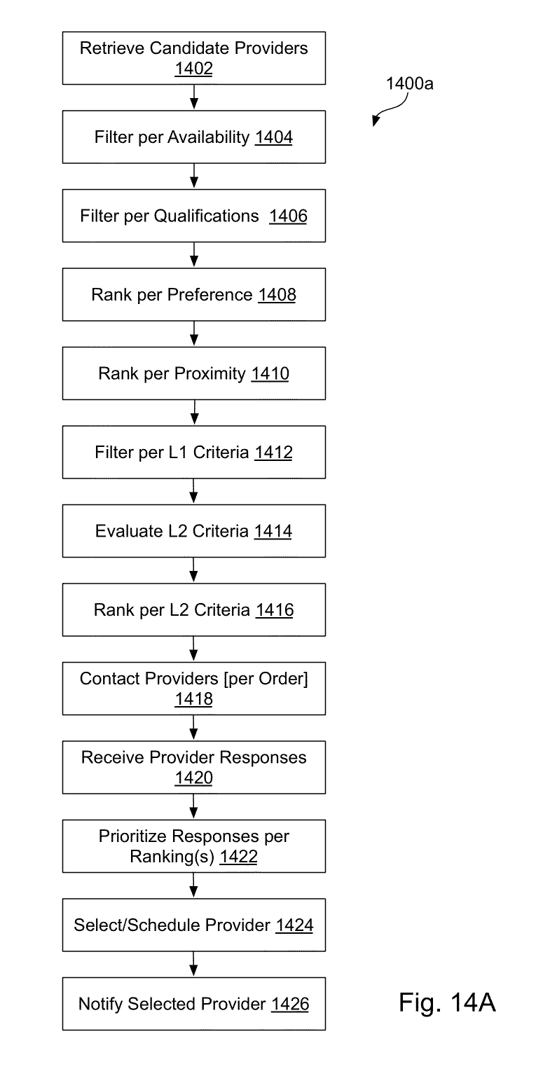

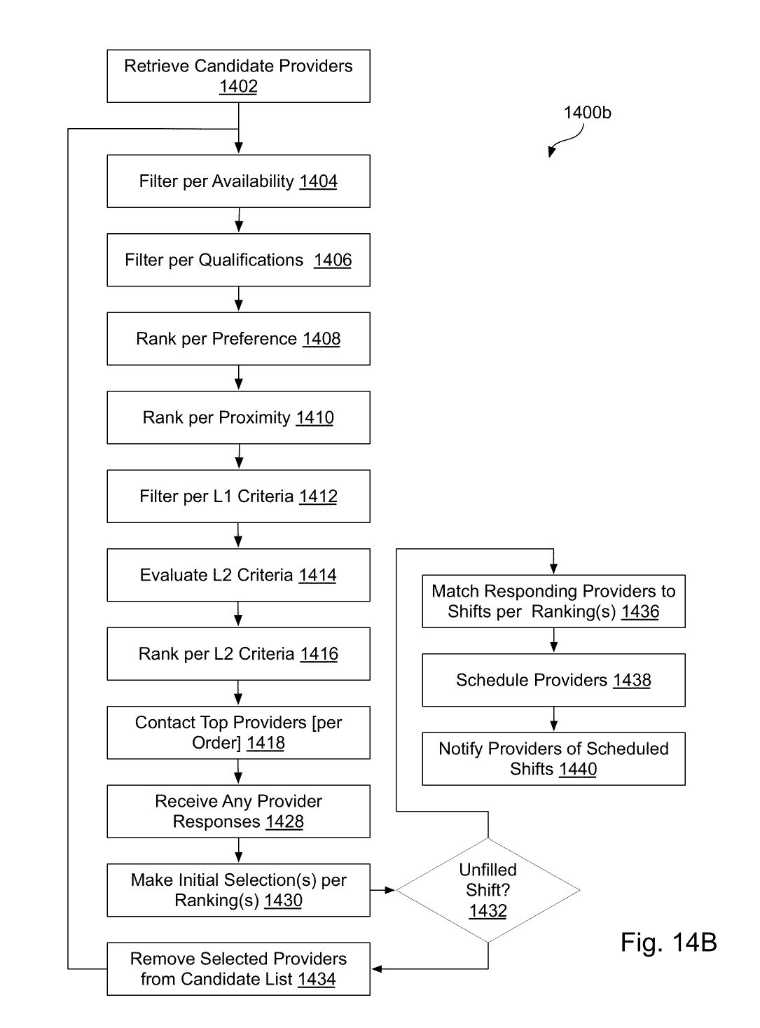

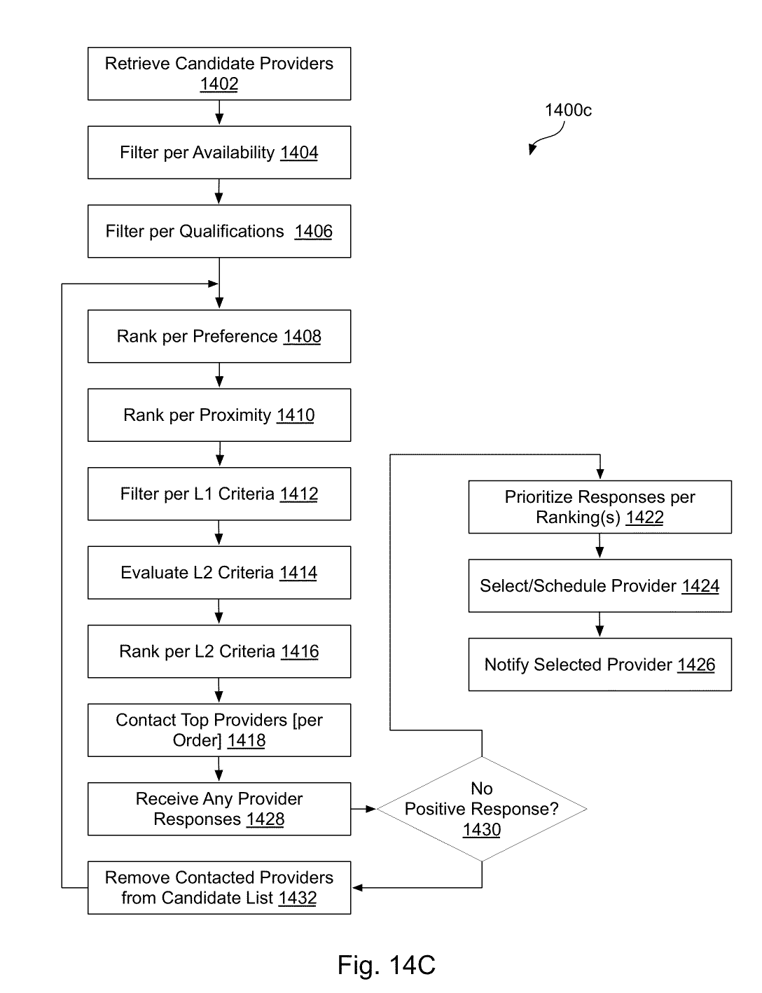

Referring to FIGS. 14A-14B, a shift, defined manually or according to any automated methods as described herein, may require a provider to be associated therewith to work the shift and otherwise perform tasks associated with the shift. In some embodiments, a shift that has been manually defined or automatically defined according to methods disclosed herein may have providers associated with according to automation methods such as are described in FIGS. 14A-14B. The methods 14A-14B presume that a shift has been defined that has at least a date associated therewith. The shift may also have start and end times, a recipient, one or more tasks, or any other information described herein as being relevant to a shift associated therewith. The methods 14A-14B may be invoked manually or automatically according to an employer preference. Various other aspects of the methods 14A-14B may be adjusted or altered according to employer preference as described hereinbelow.

Referring specifically to FIG. 14A, a method 1400a for associating a provider with a shift may include retrieving 1402 candidate providers. Candidate providers may be employees or independent contractors known to an employer or service provider. The original candidate providers may be filtered 1404 according to availability. For example, if a candidate provider is known to be scheduled at the same date or time as the shift, then that candidate provider may be removed from consideration for scheduling for the shift.

The shift for which a provider is to be scheduled may have requirements associated therewith, including a requirement that a provider working the shift have one or more certifications or qualifications. Accordingly, the candidate providers may also be filtered 1406 according to these one or more qualifications or certifications.

In some embodiment, preferences may also be imposed on selection of a provider for a shift. For example, the shift may have a recipient associated therewith and an employer may impose a preference that if possible a provider is chosen that has previously worked for that recipient. Accordingly, the method 1400a may additionally include ranking 1408 according to preference. According to the ranking 1408, candidate providers that meet more preferences or have higher scores with respect to a preference may be ranked more highly.

The preferences used for the ranking 1408 can include any arbitrary criteria. For example, preferences may include non-critical attributes that may facilitate interaction with a recipient such as gender, personality type, hobbies, personal interests, and the like.

The method 1400a may additionally include ranking 1410 providers with respect to proximity to a location associated with the shift. For example, where the shift is for providing in-home care, the candidate providers may be ranked 1410 according to proximity to the recipient's home. In some embodiments, the method 1400a may be executed with respect to multiple shifts simultaneously. Accordingly, ranking 1410 may include performing a matching step wherein candidate providers are distributed among shifts effective to one or both of reduce the total mileage required of the providers and reduce the mileage required of any individual provider. Accordingly, the ranking 1410 may also reduce the candidate providers to a group selected according to proximity according to the matching process involving multiple providers and multiple shifts.

The method 1400a my additionally include filtering 1412 candidate providers according to “level one” or “L1” criteria. L1 criteria may include skills or abilities that a provider must have in order to perform tasks associated with the shift. Examples of L1 criteria include ability to monitor or manage medical systems or conditions. For example, L1 criteria may include the ability to manage a feeding tube, perform bed transfers, manage colostomies, deal with dementia, care for wounds, provide hospice care, or care for bedbound patients. Any candidate provider that fails to meet all of the L1 criteria may be removed from consideration for a shift during the filtering step 1412.

Candidate providers may be evaluated 1414 with respect to “level two” or L2 criteria. L2 criteria may include skills or attributes that are desirable in order to perform tasks associated with a shift but not required. For example L2 criteria may include the ability to feed a patient, bath or wash a patient, dress or groom a patient, walk with a patient, assist a patient with a mobility aids, assist a patient with using the toilet, assist a patient with the patient's gait, and dealing with patients at risk of falling. Other L2 criteria may also include providing companionship, cooking and meal preparation, light housekeeping, out-of-home activities, providing car transportation, taking a patient to appointments and errands, assisting a patient with light exercise, monitoring a patient that tends to wander, and providing medication reminders. Depending on employer preference any of the exemplary L1 criteria may be treated as L2 criteria and vice versa.

A candidate provider may be ranked 1416 according to satisfaction of any of the L2 criteria. In one example, a score may be assigned to a provider according to the number of L2 criteria that have been met. In another example, L2 criteria may have different weights and a sum of the weights of all L2 criteria met by a provider may be summed to generate an L2 score for that provider.

In some embodiment, ranking 1416 according to L2 criteria may be accompanied by a filtering step wherein candidate providers that have an L2 score for a shift that is below a threshold are removed from consideration for the shift. Alternatively the top N providers with the highest L2 score may be chosen for consideration, where N is an arbitrary integer, and the rest removed from consideration.

Some or all of the candidate providers remaining after the foregoing filtering steps and as ranked according to the foregoing ranking steps may be contacted 1418 and offered the opportunity to work the shift. As noted, the method 1400a may include evaluating multiple providers with respect to multiple shifts. Accordingly, those providers that remain after filtering with respect to a specific shift may be contacted with the opportunity to work that shift. Contacting may be by means of email, text message, or automated voice telephone call to the provider's phone. Contacting 1418 may include providing some or all of the information defining the shift such as a date start time, end time, duration, recipient, address, tasks, and the like.

In come embodiments or modes of operation, candidate providers may be contacted 1418 in order of ranking. For example, a shift may be offered to providers determined suitable according to the method described above in order until a provider accepts the shift. Contact may be made in a sequential fashion in order of ranking with a delay between each attempt to contact a provider to provide an opportunity to response if a response is not received upon contact.

In some embodiments or modes of operation, candidate providers deemed suitable according to the above-described steps may be contacted 1418 substantially simultaneously. Substantially simultaneous contacting 1418 may include contacting the candidate providers at a speed allowable according to limitations of the communication means and computers performing the contacting. For example, contacts separated by less than 1 minute, preferably less than 10 seconds, and more preferably less than 1 second, may be deemed to be substantially simultaneous.

The candidate providers contacted 1418 may include all providers that remain after the above-described filtering steps. The candidate providers contacted 1418 may include less than all of the candidate providers that remain after the above-described filtering steps. For example, the top N candidate providers as ranked according to some or all of the foregoing ranking steps may be selected for contacting, with N being any predefined value.

As described above, a number of ranking steps may be included. The various rankings may be combined according to various means. For example, a score may be assigned to each caregiver as a result of each ranking steps. All the scores for the provider according to some or all of the ranking steps may then be summed to yield a final sum. The scores from different rankings may be weighted prior to summing. The final sum may then be used as a ranking when selecting the top N providers. The ranking and filtering steps of the method 1400a can be performed in any order with input to each step being candidate providers or candidate providers as reduced according to previous filtering step.

The method 1400a may include receiving 1420 any responses from the contacted providers. The responses may be received 1420 in the same manner as the providers were contacted 1418 or in a different manner. For example, responses may be received 1420 as text messages, emails, voice messages or key presses over a telephone connection, or any other means of electronic communication.

In instances where multiple providers were contacted 1418 with respect to the same shift, it is possible that multiple acceptances of the shift may be received 1420. For example, after the providers are contacted 1418, responses may be accepted within a predefined time window. All of the responses received 1420 in this window may be prioritized 1422 according to a ranking. The ranking used to prioritized the responses may be the same or different than the ranking steps described above. For instance less than all of the rankings described above may be used to rank the responding providers. A responding provider may be selected 1424 for the shift according to the ranking and associated with the shift. For example, the most highly ranked provider may be selected 1424. The selected provider may then be notified 1426 that the provider has been scheduled for the shift. Notification 1426 may be constrained to be by means of the same medium by which the selected provider responded to the invitation. Notification 1426 may be by means of text message, voice message, email, or any other electronic messaging means.

In an alternative embodiment or mode of operation, shifts may be scheduled on a first-come first-served basis such that the first provider to respond is selected and scheduled for the shift regardless of ranking. In some embodiments or modes of operation, the selected provider may be presented to a supervisor or other member of management for approval and approval received prior to actual scheduling of the provider for the shift and notification of the provider. Where approval is not received, an alternative selection may be received or an alternative candidate may be selected and presented for approval, such as the next lowest ranked provider.

FIG. 14B illustrates an alternative method 1400b for associating providers with shifts. The steps 1402 through 1418 may be as described hereinabove. The method 1400b has particular application to modes of operation wherein multiple providers are being invited to fill multiple shifts. Accordingly steps 1402 through 1418 may be executed with respect to multiple shifts.

For example, a common pool of providers may be processed independently for each shift in order to perform filtering steps 1404, 1406 with respect to requirements specific to each shift. Thereafter each shift may have a group of candidate providers suitable for the shift. In some embodiments, steps 1408-1416 may also be performed independently for each of these groups. Alternatively, the steps 1408-1416 may be performed in an interdependent manner to arrive at mutually exclusive groups of candidate providers for each shift. As an example, the steps 1408-1416 may be performed for each shift using the group of candidate providers output from the filtering steps 1404-1406 for the shift. Prior to contacting 1418 the top providers, an optimization or matching step may be performed to generate mutually exclusive groupings of candidate providers for each shift. Any optimization routine may be used. For example, as noted above, ranking may result in a score associated with respect to each provider with respect to each shift. Accordingly, the output of the optimization or matching step may result in mutually exclusive groupings of providers for each shift such that the providers in each grouping yield the highest possible cumulative score for that shift.

Optimization of the groupings may also take into account individual rankings 1408, 1410, 1416 and each of these rankings may be given different weight according to employer or developer preference. The cost function or other metric that is minimized or maximized according to the optimization algorithm may be a function of some or all of these ranking outputs or a combination of some or all of these rankings.

Once groupings of candidate providers have been determined for each shift, these candidate providers may then be contacted 1418 as described above. The contact may include information about the shift for which the candidate providers were selected as a suitable as described above. Any responses to the contact 1418 may be received 1428. An initial selection of a provider for each shift may then be performed 1430 with respect to each shift for which any acceptances were received. The initial selection 1430 may occur at a specified time interval after contact. Where no response was received or no acceptances of invitations were received with respect to a shift, then no selection may be made 1430.

If shifts are found 1432 to be unfilled, then providers that were selected 1434 may be removed from the pool of candidate providers. The method 1400b may then repeat at any point in the process, such as with ranking 1408 according to preference. In an alternative embodiment, the process may repeat prior to one or both of the filtering steps 1404-1406.

Processing may then repeat as described until all of the shifts are found 1432 to have been filled. Alternatively, the process may repeat until one of a maximum number of iterations have occurred and all the shifts are filled. Once an initial selection has been made with respect to each shift, a final matching 1436 or optimization step may occur. This may include evaluating the availability indicated by each of the selected providers and possible changing the initial selection such that a provider selected for one shift may be assigned to a different shift. The optimization or matching criteria may include some or all of the ranking criteria described above. For example, the matching algorithm may adjust assignments of providers to shifts in order to minimize driving distance or to ensure the driving distances of each provider are as small as possible.

The final assignments of providers to shifts as determined by the matching or optimization step 1436, the providers may then be scheduled 1438 for the shifts according to the final assignments. The providers selected for each shift may then be notified 1440 of the final assignment. Notification may be according to any of the means of communication described herein.

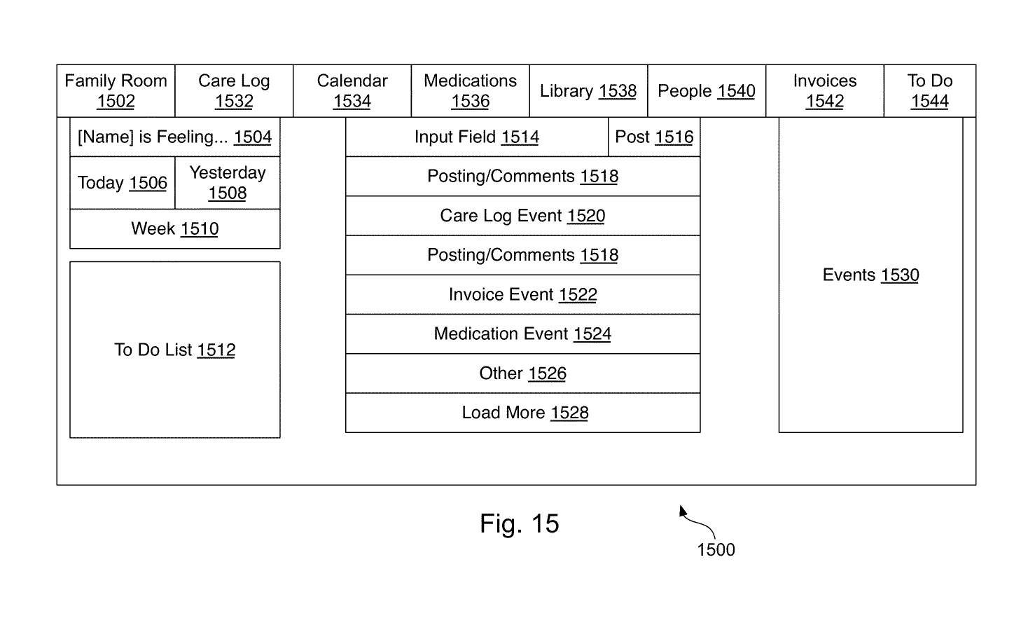

FIG. 15 illustrates a family portal 1500 that may be implemented along with the interfaces and methods described hereinabove. For purposes of this disclosure, the interfaces and methods of FIGS. 1-14B are considered to be enterprise interfaces and methods, collectively referred to herein as an enterprise portal, that are accessed or otherwise used by an enterprise to manage care provided by the employees and representatives of the enterprise. In contrast, the family portal 1500 allows family members or other concerned individuals of a recipient to view and provide input regarding the condition of the recipient and care provided to the recipient. For purposes of this disclosure a recipient can be a patient receiving medical care or a person or other entity receiving any other type of service or product. For purposes of this disclosure, the recipient may also perform some or all of the actions ascribed to a family member or concerned individual for the recipient. The functionality ascribed herein to the enterprise portal and family portal may be implemented by the same server computer systems or different server computer systems in data communication with one another.

For example, the family portal 1500 may include an interface element 1502 may be selectable by a user to invoke display of the illustrated family room interface regarding a recipient. The interface may illustrate various aspects of recipient care. For example, the family room interface may include an emotional state element 1504 operable to display a current emotional state of a recipient such as “[name] is feeling [state],” where state is an indicator of an emotional state provided either by the recipient or a caregiver based on observation of the recipient. In some embodiments, a computer system may receive through an enterprise portal, an indicator of a recipient's emotional state and associate this with one or more records associated with the recipient and a record of the shift and/or task for which the indicator was received. For example, a task associated with a shift, such as according to any of the methods disclosed herein, may include a task to input or otherwise obtain and input an indicator of the emotional state of the recipient at some point in the shift, such as at the beginning and/or end. In some embodiments, an input indicator of an emotional state is a short video of the recipient stating the recipient's current mood or pain level. This video may then be used as the indicator. In some embodiments, this video may be analyzed by a computer algorithm to obtain an estimate of the recipient's condition, such as based on facial expressions of the recipient. In some embodiments, the indicator may be a selection of a word from a list of words in an interface presented to the provider or a symbol (e.g. smiley face, frowning face, or some other symbol).

The emotional state element 1504 may include indicators 1506, 1508, 1510, for emotional states of the recipient recorded at various past times, such as “today,” “yesterday,” the preceding week, month, or any other time interval.

In some embodiments, the family room interface may include a to do list 1512 that lists tasks that may be tasks not associated with the shift of a provider. The tasks may be input by a concerned individual through the interface 1500 and may include tasks input through the enterprise portal by administrators, health care providers, doctors, and other personnel. The to do list 1512 may include tasks with or without a date associated therewith. The items of the to do list 512 may further include a completion status (to do, in progress, done, etc.). The completion status for tasks input through the enterprise portal may be updatable only through the enterprise portal. Likewise, the completion status for tasks in the to do list 512 may be updatable only through the family portal, such as only by the individual that created the task.

The family room interface may additionally include a feed of events. The events of the feed may be presented in reverse chronological order, e.g. most recent event first. As will be described in greater detail below, the events may include events received through the family portal as well as events derived from the enterprise, e.g. data obtained regarding shifts, tasks, and the status of tasks as described hereinabove with respect to FIGS. 1-14B.

The feed may be displayed in association with an input field 1514 and an interface element 1516 to invoke the addition of text, images, or other files, provided in the input field 1514 to the feed. Data posited by means of the interface element 1516 may be added to the feed having a date associated therewith equal to the date the posting was received.

Events posted by means of the input field 1514 may be added to the feed as postings 1518. As for other forums known in the art, other users may input comments to a posting through the family portal and these comments may be displayed with the postings 1518 in the interface 1500.

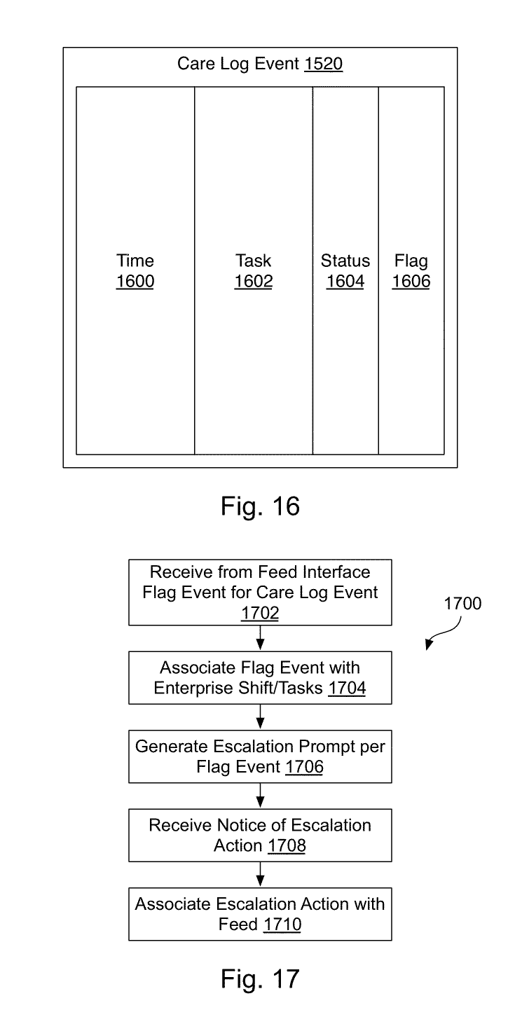

The feed may include care log events 1520. A carelog event 1520 may include a representation of the data defining a shift worked on behalf of the recipient, the tasks of the shift, and the status of the tasks of the shift as generated and updated according to the methods described herein. A detailed representation of a carelog event is shown in greater detail in FIG. 16.

The feed may include invoice events 1522. An invoice event 1522 may include a representation of a bill being sent to a payer for a recipient's care, payment of the bill, the bill coming due, the bill being past due, or some other action or event relating to payment for services to a recipient. The invoice events may be received from enterprise data for the recipient and events created for addition to the feed. In some embodiments, the events of a feed for a recipient may be stored as such as in a database. A database of enterprise data including the shift, task, and tasks status data described above may also be maintained and include any of the above invoice data. In some embodiments, an enterprise computer system may detect changes to invoice data and generate invoice events 1522 corresponding thereto. In other embodiments, a software module may periodically review the enterprise database to identify invoice events and generate corresponding invoice events.

The feed may further include medication events 1524. In an enterprise providing medical services, tasks associated with a shift or otherwise associated with a recipient may include tasks to administer medications. In some embodiments, medication events 1524 include representation of the administration of medication in accordance with such as task. In other embodiments, medication events 1524 only include changes to a list of medications administered to a recipient or changes to the schedule of doses of the medication, an amount of doses, or other change to the regimen of medications administered to the recipient. In such embodiments, a representation of actual administration of medications may be reported in carelog events 1520. As for other events in the feed, medication events 1524 may be derived from an enterprise database that records such events in order to provide care to a recipient. The actual changes to a regimen of medications for a recipient may be received through the enterprise portal and the enterprise portal updated accordingly. As for other events in the feed, these changes may be detected as they occur by a software module that generates corresponding events or the software module may periodically evaluate the enterprise database to determine changes and generate the corresponding events for addition to the feed.

The feed may additionally include one or more other events 1526 that report other aspects of care provided to a recipient or events relating to recipient care, a recipient's personal life, or the lives of family and friends of the recipient. Inasmuch as screen space may be limited, the feed may include an interface element 1528 to invoke display of additional events from the feed.

The family room interface may additionally include a listing of upcoming events 1530. The events may be personal events input through the family portal 1500 by the recipient or other concerned individuals. The events may also include events from a care schedule associated with the recipient in an enterprise database. These events may be extracted and displayed as events 1530 in the family portal 1500.

The family room interface 1500 may include various other interface elements for invoking the display of other information relating to a recipient and invoking display of interfaces for receiving information for associating with a recipient. For example, the interface 1500 may include an carelog element 1532 for invoking display of carelogs relating to a recipient. A carelog may include, for example, a display of shifts and corresponding tasks for the recipient as well as the status of these tasks. The carelog element 1532 may further invoke display of a shift schedule presenting future shift and corresponding tasks for the recipient, as defined in an enterprise database using the methods of FIGS. 1-14B.

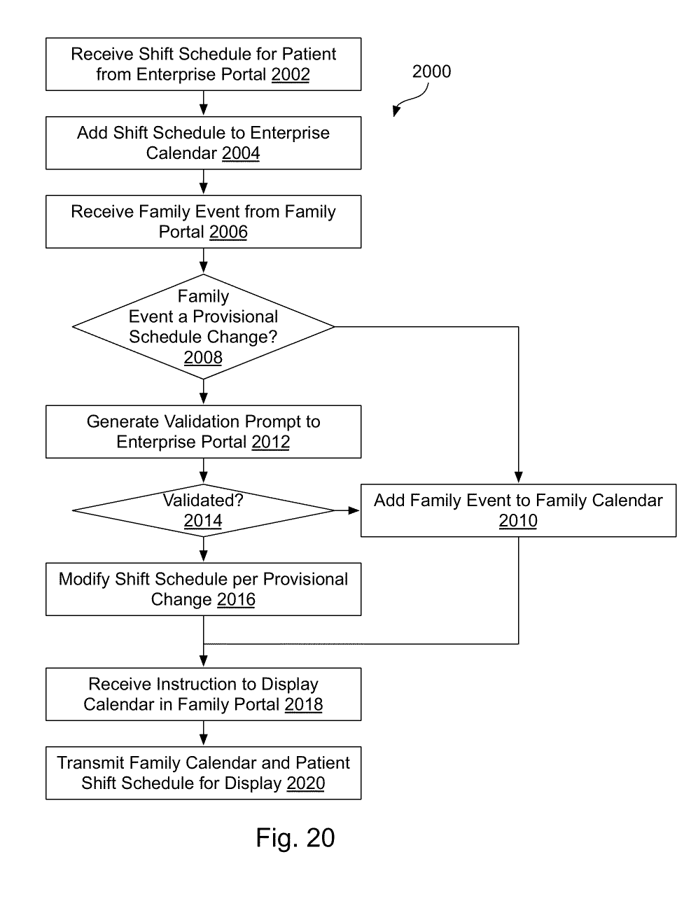

The family room interface 1500 may include a calendar interface element 1534 for invoking display of one or more calendars. For example, the calendar interface element 1534 may invoke display of an enterprise calendar displaying recipient care events schedule by the enterprise for the recipient. The calendar interface element 1534 may also invoke display of one or more personal calendars, each corresponding to a particular person or group of persons. For example, a recipient may have a personal calendar listing events relating to the recipient. Family members may also have calendars for individuals or for the family as an entity. In some embodiments, multiple calendars may be displayed simultaneously, i.e., a user of the family portal 1500 may invoke display of a calendar listing events from a plurality of calendars of the above-referenced calendars. In some embodiments, a family member or other concerned individual may make provisional changes to an enterprise calendar for the recipient, as described in greater detail with respect to FIG. 20.

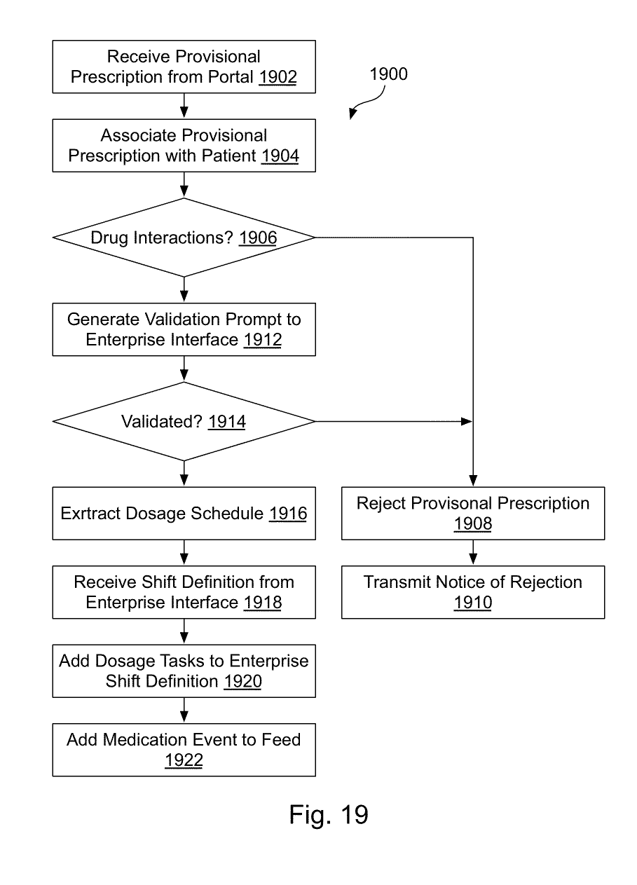

The family room interface 500 may include a medications interface element 1536 that invokes an interface for viewing by family members and concerned individuals of a recipient's medications, including some or all of medications taken, dosage schedule, dosage amount, refill dates, amount left, or any other information. In some embodiments, family members or other concerned individuals may be responsible or enabled to refill prescriptions or propose changes to medication based on prescriptions. Accordingly, element 1536 may invoke an interface enabling a user to enter, through the family portal a proposed change to a recipient's medications. A more detailed method for processing provisional changes to a recipient's medications is discussed in greater detail below with respect to FIG. 19.

A library interface element 1538 of the portal 1500 may invoke display of a library of documents associated with a recipient. The library interface element 1538 may invoke display of an interface by which a family member or concerned individual may upload documents. In some embodiments, documents in an enterprise database relating to the recipient may be accessible through the family portal 1500. Examples of documents included in a library may include such documents as contracts governing the provision of care to a recipient, photographs or videos of personal interest, medical literature relating to a recipient's condition, or any other documents of professional or personal interest.

A people interface element 1540 may invoke display of an interface for controlling access and other privileges for individual's associated with a recipient. For example, an administrator may be associated with the family portal 1500 associated with a recipient. The administrator may provide other users with access to the family portal 1500, such as associating a username, password, and privileges for the user with the data defining the family portal 1500. Examples of privileges that may be associated with a user include the ability to view the family room interface feed, the ability to post comments to the feed, the ability to make provisional changes to the enterprise calendar for the recipient, the ability to make provisional changes to medications, the ability to access sensitive medical information (e.g. information protected by the Health Information Privacy Act (HIPA)), or the ability to access or generate any type of information described herein as associated with the family portal 1500.

An invoices interface element 1542 may invoke display of invoice data from an enterprise database relating to a recipient. The invoices interface element 1542 may further invoke display of an interface enabling a family member or concerned individual to tender electronic payment for an interface. The invoice data may include such information as past paid invoices, currently due invoices, past due invoices, information regarding reimbursement for expenses by an insurance company or government agency.

A “to do” interface element 1544 may invoke display of a to do list or an interface for adding tasks to do list 1512. The tasks entered using element 1544 may include tasks for health care providers or specific individuals that are participating in taking care of the recipient using the family portal 1500.

FIG. 16 illustrates an example illustration of a care log event 1520. A care log event 1520 may correspond to a shift worked by a provider with respect to the recipient, such as a shift defined according to the methods of FIGS. 1-14B. The care log event 1520 may include a time column 1600 and a task column 1602 that list the time a task associated with the shift was completed and brief title or description of the task, respectively. The care log event 1520 may additionally include a status column 1604 that lists a status of each task, e.g. completed, not completed, partially completed, or some other metric or indicator of a completion status of a task, such as a status reported according to the methods described herein.

The care log event 1520 may include a flag column 1606 that lists any flags associated with each task of a shift. The flag column 1606 may include interface elements for each task that, when selected by a user, invokes an interface whereby a family member or concerned individual can associate a flag with the task. For example, an interface for inputting a flag may present a predefined list of possible grievances or provide an input field whereby a user may specify a concern. Where a user has previously specified a flag, the flag may be represented in the flag column 1606, such as by means of a symbol, text, or some other graphical representation.

FIG. 17 illustrates a method 1700 that may be executed using a family portal 1500 including care log events 1520, such as care log events 1520 in an event feed. The method 1700 may be executed by a server computer system providing a family portal 1500 and access to the family portal 1500. The method 1700 may include receiving 1702 a flag event for a care log event, such as a task of a care log event. This may include receiving a selection of a flag input element in a flag column 1606 of a care log event and receiving a user selection or input of a description of a concern or other commentary on the task.

The method 1700 may further include associating 1704 the flag event with a record of the shift in an enterprise database, such as by storing the concern input by the user with the record of a shift corresponding to the care log event, where the shift is a shift generated and updated according to any of the methods described with respect to FIGS. 1-14B.

In response to the flag event, the method 1700 may include generating 1706 a prompt, through the enterprise portal, to take action with respect to the flag. For example, the concern associated with the flag event may be displayed in, or accessed through, any of the interfaces described hereinabove for inputting or otherwise accessing a record of a shift and the tasks thereof. In some embodiments, the prompt may be actively output to a user accessing the enterprise portal without requiring the user to request access to information relating to the shift. In other embodiments, the prompt may be a visual indicator associated with a graphical representation of the shift when accessed by the user, even if for some other reason.

The method 1700 may further include receiving 1708, through the enterprise portal, notification of an escalation with respect to the flag event. Escalation may include investigation of the concern by a supervisor, a notice to the provider associated with the shift of the concern from the flag, generating one or more tasks or reminders to a supervisor to follow up with respect to the task or type of task associated with the flag event. For example, a supervisor may have a calendar associated therewith and an escalation event may include adding reminders to the calendar to follow up with respect to the task or type of task.

The method may further include associating the escalation action with the feed accessible through the family portal 1500. As noted above, the care log event may be a separate data structure associated with the event feed for the recipient accessible through the family portal 1500. Accordingly, the care log event stored in the feed may be modified to record the escalation action associated with the flag event. This may include associating in the data defining the feed a record of the escalation using a data structure recording the original flag event received at step 1702. Upon subsequent access to the family portal 1500 a viewer of the feed will then be presented with a care log event 1520 that includes a record of the escalation event or an interface element for invoking display of the escalation event.

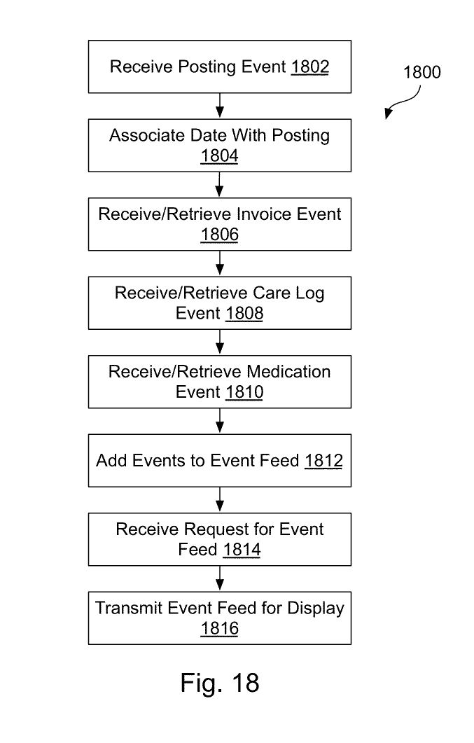

FIG. 18 illustrates a method 1800 that may be executed by the server system or server systems implementing an enterprise portal and family portal 1500. The method 1800 may include receiving by the family portal 1500 one or more events. This may include receiving 1802, by the family portal 1500, a posting event. A posting event may be received 1802 from a web browser executing on a user computer of a user having privileges to make postings to the event feed. As noted above, a received 1802 posting may include text, images, video files, audio files, or any other content or object that may be usable by users, such as content or objects that can be rendered or otherwise played back by a browser.

The method 1800 may include receiving 1806 or retrieving, by the family portal from the enterprise portal, or some other software module accessing an enterprise database, one or more invoice events from the enterprise database. An invoice event may include any of the invoice events described hereinabove.

The method 1800 may include receiving 1808 or retrieving, by the family portal from the enterprise portal, or some other software module accessing an enterprise database, a care log event, such as a care log event as described hereinabove.

The method 1800 may include receiving 1810 or retrieving, by the family portal from the enterprise portal, or some other software module accessing an enterprise database, a medication event, such as a medication event as described hereinabove.

The one or more events received 1806-1810 may then be added 1812 to an event feed. Adding 1812 an event to an event feed may include creating a data structure including the data defining the event and may include storing formatting data for displaying the data in a web browser or other interface. In some embodiments, adding 1812 an event to the event feed may include adding a link or pointer referencing the data defining the event to the data defining the event feed, such as a location of the data in the enterprise database, rather than storing the actual data defining the event in the event feed.

The method 1800 may further include receiving 1814 a request for the event feed. The request may be receive through the family portal and may be embodied as a request for a URL (uniform resource locator) associated with the family portal 1500 or a particular recipient account accessible through the family portal. In some embodiments, a request may be generated by a web page accessible through the family portal 1500, such as by a user logging on using the web page to an account linked with the account of the recipient for which the requested feed is maintained.

In response to the received 1814 request, the family portal may transmit 1816 some or all of the event feed for display on a user computing device. For example, the most recent N events in the event feed may be transmitted 1816 for display. Transmitting 1816 the events may include formatting the events for rendering in a web browser or some other interface executing on the user computing device.

FIG. 19 illustrates a method 1900 that may be executed by a family portal 1500, such as a server system implementing the family portal 1500. The method 1900 may include receiving 1902, through the family portal 1500 a provisional change to the prescriptions of a recipient. The provisional change may be input, on a user computing device, to an interface to the family portal 1500. The provisional change may be in response to a prescription by a doctor obtained by a family member of the recipient that is helping to coordinate the recipient's care, such as by taking the recipient to doctor visits.

The method 1900 may further include associating 1904 the provisional change with the recipient in an enterprise database. For example, the provisional prescription change may be stored with data defining prescriptions of the recipient. Prescriptions and provisional prescriptions may include such information as a medication name, dosage schedule, refill date, start date, end date, or other data characterizing a prescription. A provisional prescription may be flagged as such in the record storing it.

The method 1900 may further include evaluating 1906 the provisional prescription change with respect to existing prescriptions of the recipient to determine whether there are any known adverse drug interactions or allergies of the recipient with respect to the provisional prescription change. Where the provisional prescription change is the removal of a medication, then the evaluation 1906 may be omitted. Evaluating adverse drug interactions may include evaluating literature defining such interactions, such as provided by the Food and Drug Administration (FDA).

Where an adverse drug interaction or known allergy is found 1906 to be present, the method 1900 may include rejecting 1908 the provisional prescription change, e.g., deleting a record of the provisional prescription change in the enterprise database or otherwise flagging the provisional prescription change as rejected or in need of closer review. In some embodiments, a notice of rejection may be transmitted 1910 to the family portal 1500 for display to users interacting with the family portal 1500. The notice may also be transmitted to the user that input the provisional prescription change, such as by means of email or message retrieved through the family portal 1500. In some embodiments, a rejection of a provisional prescription change may be transmitted 1910 by creating an event in an event feed as described herein.

Where no drug interaction or allergy is found 1906, the method 1900 may include generating 1912 a prompt through the enterprise portal to validate or reject the provisional prescription change. The prompt may be an alert output to an enterprise representative, such as a health care professional, accessing an interface of the enterprise portal on an enterprise computing device. Alternatively or additionally, the alert may be passively displayed to an enterprise representative viewing display of a recipient's prescriptions on the enterprise computing device.

The method 1900 may further include evaluating 1914 whether validation or rejection of the provisional prescription has been received. In some embodiments, a provisional prescription for which validation is not received within N days may be found 1914 to have been rejected. A provisional prescription may be validated or rejected by means of an instruction communicating one of these outcomes received by the enterprise portal from an enterprise computing device. Where provisional prescription is found 1914 to have been rejected, the method 1900 may include performing one or both of steps 1908 and 1910 as described hereinabove.

Where a provisional prescription change is found 1914 to have been validated, the method 1900 may include creating tasks corresponding to the dosage requirements of the provisional prescription. Where the provisional prescription removes a prescription, then tasks corresponding to this prescription may be removed from a care schedule for the recipient, such as by removing corresponding dosing tasks in shifts of providers.

Where the provisional prescription change is a new prescription or a change to an existing prescription, the method 1900 may include extracting 1916 a dosage schedule from the prescription. For subsequently received 1918 shift definitions for the recipient, the method 1900 may include adding 1920 dosing tasks to the shifts according to the extracted 1916 dosing schedule. The dosing tasks may then be processed and updated according to the methods described hereinabove with respect to the methods of FIGS. 1-14B. The method 1900 may further include adding 1922 a medication event to the event feed of the recipient that is accessible through the family portal. A medication event may be added 1922 the informs of the validation of the provisional prescription change along with the parameters defining the prescription change.

In some embodiments, events such as tasks, tasks on a to-do list, or reminders in an enterprise calendar may also be generated to refill the provisional prescription after it has been validated in accordance with a refill schedule defined by the provisional prescription.

Referring to FIG. 20, in some embodiments, an authorized user may make provisional changes to an enterprise calendar for a recipient through the family portal 1500. As an example, where the enterprise provides in-home care, a family member may perform these tasks during an extended visit. A provisional change to the calendar may inform the enterprise of this fact.

The method 2000 of FIG. 20 may be used to propagate provisional changes to an enterprise calendar received through the family portal to an enterprise calendar. The method 2000 may include receiving 2002 through the enterprise portal a shift schedule for a recipient. The shift schedule may include any of the parameters defining a shift and the tasks thereof described hereinabove and the shift schedule may be defined using any of the interfaces and methods described hereinabove. The shift schedule may be added 2004 to an enterprise calendar for the recipient and stored in an enterprise database.

The method 2000 may further include receiving 2006 through the family portal 1500 family events, such as from a user computing device interfacing with the family portal 1500. The event may have a date associated therewith and may be a personal event or a provisional change to an enterprise calendar for the recipient, such as a shift schedule for providers providing care to the recipient.

If the family event is not found 2008 to be a provisional change to a shift schedule, the event may be added 2010 to a family calendar associated with the calendar, which may include one of multiple calendars for personal events for the recipient and family members of the recipient. A provisional change may be the cancelation of shifts, a proposed addition of shifts, a change in the duration of shifts, or a change to specific tasks of shifts.

If the family event is found 2008 to be a provisional change to the shift schedule, the method 2000 may include generating 2012 a prompt issued through the enterprise portal to validate or reject the provisional change. As for other prompts disclosed herein, the prompt may be actively displayed and output, such as in the form of a pop up window, audible alert or the like. The prompt may also be a passive indicator in a display of the shift schedule accessed through the enterprise portal, where the passive indicator communicates a need to validate a proposed change to the shift schedule.

If validation of the provisional change is not found 2014 to have occurred, the provisional change may simply be ignored or deleted. In some embodiment, the provisional change may be preserved as an event added 2010 to a family calendar, but not be the basis of a change to an enterprise shift schedule. A provisional change may be found 2014 to be rejected based on an explicit rejection received through the enterprise portal, such as from an enterprise computing device communicating with the enterprise portal. A provisional change may also be found 2014 to be rejected if the date associated therewith passes without having received validation. A provisional change may also be found 2014 to be rejected if N days pass without validation having been received after the provisional change is received 2006. In other embodiments, the method 2000 may be biased in favor of acceptance, such that a provisional change will be found 2014 to be validated if a rejection thereof is not received with N days of receipt 2006.

If validation is found 2014 to have occurred, the method 2000 may include modifying a shift schedule in the enterprise database in accordance with the provisional change. Thereafter, instructions may be received to display the calendar through the family portal 1500. In response thereto, the family calendar as updated according to any received 2006 family events may be transmitted 2020 for display on a requesting user computing device. A shift schedule for providers providing care to the recipient may also be transmitted 2020 for display. Where the received 2006 event has been validated, a shift schedule transmitted for display may reflect the change to the shift schedule in accordance with the event.

As discussed herein, the invention may involve a number of functions to be performed by a computer processor, such as a microprocessor. The microprocessor may be a specialized or dedicated microprocessor that is configured to perform particular tasks according to the invention, by executing machine-readable software code that defines the particular tasks embodied by the invention. The microprocessor may also be configured to operate and communicate with other devices such as direct memory access modules, memory storage devices, Internet-related hardware, and other devices that relate to the transmission of data in accordance with the invention. The software code may be configured using software formats such as Java, C++, XML (Extensible Mark-up Language) and other languages that may be used to define functions that relate to operations of devices required to carry out the functional operations related to the invention. The software code may also include scripting languages such Pearl, Python, PHP, and the like. The code may be written in different forms and styles, many of which are known to those skilled in the art. Different code formats, code configurations, styles and forms of software programs and other means of configuring code to define the operations of a microprocessor in accordance with the invention will not depart from the spirit and scope of the invention.

Within the different types of devices, such as laptop or desktop computers, hand held devices with processors or processing logic, and also possibly computer servers or other devices that utilize the invention, there exist different types of memory devices for storing and retrieving information while performing functions according to the invention, this is used for transitive and non-transitive storage. Cache memory devices are often included in such computers for use by the central processing unit as a convenient storage location for information that is frequently stored and retrieved. Similarly, a persistent memory is also frequently used with such computers for maintaining information that is frequently retrieved by the central processing unit, but that is not often altered within the persistent memory, unlike the cache memory. Main memory is also usually included for storing and retrieving larger amounts of information such as data and software applications configured to perform functions according to the invention when executed by the central processing unit. These memory devices may be configured as random access memory (RAM), static random access memory (SRAM), dynamic random access memory (DRAM), flash memory, and other memory storage devices that may be accessed by a central processing unit to store and retrieve information. During data storage and retrieval operations, these memory devices are transformed to have different states, such as different electrical charges, different magnetic polarity, and the like. Thus, systems and methods configured according to the invention as described herein enable the physical transformation of these memory devices. Accordingly, the invention as described herein is directed to novel and useful systems and methods that, in one or more embodiments, are able to transform the memory device into a different state during transitive and non-transitive storage. The invention is not limited to any particular type of memory device, or any commonly used protocol for storing and retrieving information to and from these memory devices, respectively.

Although the components and modules illustrated herein are shown and described in a particular arrangement, the arrangement of components and modules may be altered to process data in a different manner. In other embodiments, one or more additional components or modules may be added to the described systems, and one or more components or modules may be removed from the described systems. Alternate embodiments may combine two or more of the described components or modules into a single component or module.

Finally, although specific embodiments of the invention have been described and illustrated, the invention is not to be limited to the specific forms or arrangements of parts so described and illustrated. The scope of the invention is to be defined by the claims appended hereto, any future claims submitted here and in different applications, and their equivalents.

The foregoing description has been presented for the purposes of illustration and description. It is not intended to be exhaustive or to limit the invention to the precise form disclosed. Many modifications and variations are possible in light of the above teaching. Further, it should be noted that any or all of the aforementioned alternate embodiments may be used in any combination desired to form additional hybrid embodiments of the invention.

What is claimed is:

1. A method for managing provision of healthcare, the method comprising:
receiving, by a computer system through a family portal, a posting to an event feed for a recipient, the posting including at least one of text and a picture;
associating, by the computer system, a posting event including with the event feed for the recipient;
receiving, by a computer system through an enterprise portal, a definition of a shift including a plurality of tasks performed on behalf of the recipient and a provider;
receiving, by the computer system through the enterprise portal, updates to a status of the plurality of tasks;
updating, by the computer system, the shift according to the received updates;
posting, by the computer system, a report of the shift and the status of the plurality of tasks to the event feed; and
transmitting, by the computer system for access through the family portal, the event feed.

2. The method of claim 1, further comprising:
receiving, by the computer system through the family portal, interaction with a graphical representation of the shift in a display of the event feed, the interaction specifying a flag with respect to a task of the plurality of tasks; and
associating, by the computer system, the flag with the task;
outputting through the enterprise portal, by the computer system, a status of the task according to the flag.

3. The method of claim 2, further comprising:
receiving, by the computer system through the enterprise portal, a response to the flag; and
updating, by the computer system, the report of the shift in the event feed in accordance to the response.

4. The method of claim 1, further comprising:
receiving, by the computer system through the enterprise portal, notification of tender of payment for an invoice for the recipient; and
posting, by the computer system, an invoice event reporting the tender of payment to the event feed.

5. The method of claim 1, further comprising:
receiving, by the computer system through the enterprise portal, an update to prescription medications for the recipient; and
posting, by the computer system, a prescription event reporting the update to the prescription medications to the event feed.

6. The method of claim 1, further comprising, receiving, by the computer system through the family portal, a comment to the posting event and associating the comment with the posting event.

7. The method of claim 1, further comprising:
receiving, by the computer system through the enterprise portal, a notification of a current emotional state of the recipient; and
associating, by the computer system, a emotional state event indicating the current emotional state with the event feed.

8. The method of claim 1, further comprising:
receiving, by the computer system through the enterprise portal, a non-event task from a medical professional assigned to the recipient; and
associating, by the computer system, the non-event task with the event feed.

9. The method of claim 1, further comprising:
receiving, by the computer system through the enterprise portal, a non-event task from a medical professional assigned to the recipient; and
associating, by the computer system, the non-event task with the event feed.

10. The method of claim 1, further comprising:
receiving, by the computer system through the family portal, a modification to an enterprise shift calendar for the recipient;
associating, by the computer system, the modification as a provisional change to the enterprise shift calendar;
outputting, by the computer system through the enterprise portal, a prompt to validate the provisional change; and
if validation is received, modifying, by the computer system, the enterprise shift calendar in accordance with the provisional change.

11. The method of claim 1, further comprising:
receiving, by the computer system through the family portal, a new prescription for the recipient;
verifying, by the computer system, a lack of adverse interactions of the new prescription with respect to current prescriptions for the recipient;
if no adverse interactions are found, outputting, by the computer system through the enterprise portal, a prompt to validate the new prescription; and
if validation is received, adding, by the computer system, dosaging tasks in accordance with a dosage schedule for the new prescription to the shift.

12. A system for managing provision of healthcare, the system comprising one or more processors and one or more memory devices operably coupled to the one or more processors, the one or more memory devices storing executable and operational data effective to cause the one or more processors to:
implement a family portal and an enterprise portal;
receive, through the family portal, a posting to an event feed for a recipient, the posting including at least one of text and a picture;
associate a posting event including the posting with the event feed for the recipient;
receive, through the enterprise portal, a definition of a shift including a plurality of tasks performed on behalf of the recipient and a provider;
receive, through the enterprise portal, updates to a status of the plurality of task;
updating the shift according to the received updates;
post a report of the shift and the status of the plurality of tasks to the event feed; and
transmitting, through the family portal, the event feed.

13. The system of claim 1, wherein the executable and operational data are further effective to cause the one or more processors to:
receive, through the family portal, interaction with a graphical representation of the shift in a display of the event feed, the interaction specifying a flag with respect to a task of the plurality of tasks; and
associate the flag with the task;
output, through the enterprise portal, a status of the task according to the flag.

14. The system of claim 13, wherein the executable and operational data are further effective to cause the one or more processors to:
receive, through the enterprise portal, a response to the flag; and
update the report of the shift in the event feed in accordance to the response.

15. The system of claim 12, wherein the executable and operational data are further effective to cause the one or more processors to:
receive, through the enterprise portal, notification of tender of payment for an invoice for the recipient; and
post an invoice event reporting the tender of payment to the event feed.

16. The system of claim 12, wherein the executable and operational data are further effective to cause the one or more processors to:
receive, through the enterprise portal, an update to prescription medications for the recipient; and
post a prescription event reporting the update to the prescription medications to the event feed.

17. The system of claim 12, wherein the executable and operational data are further effective to cause the one or more processors to receive, through the family portal, a comment to the posting event and associating the comment with the posting event.

18. The system of claim 12, wherein the executable and operational data are further effective to cause the one or more processors to:
receive, through the enterprise portal, a notification of a current emotional state of the recipient; and
associate an emotional state event indicating the current emotional state with the event feed.

19. The system of claim 12, wherein the executable and operational data are further effective to cause the one or more processors to:
receive, through the family portal, a modification to an enterprise shift calendar for the recipient;
associate the modification as a provisional change to the enterprise shift calendar;
output, through the enterprise portal, a prompt to validate the provisional change; and
if validation is received, modify the enterprise shift calendar in accordance with the provisional change.

20. The system of claim 12, wherein the executable and operational data are further effective to cause the one or more processors to:
receive, through the family portal, a new prescription for the recipient;
verify a lack of adverse interactions of the new prescription with respect to current prescriptions for the recipient;
if no adverse interactions are found, output, through the enterprise portal, a prompt to validate the new prescription; and
if validation is received, add dosaging tasks in accordance with a dosage schedule for the new prescription to the shift.
